(19) United States
(12) Patent Application Publication
(10) Pub. No.: US 2026/0002152 A1
(43) Pub. Date: Jan. 1, 2026

(54) Antisense Nucleic Acids for Use in the Treatment for LMNA Mutation Carriers

(71) Applicant: Stichting Amsterdam UMC, Amsterdam (NL)

(72) Inventors: Yigal-Martin Pinto, Utrecht (NL); Anke Johanna Marina Tijsen, Kockengen (NL); Dylan Kristanto De Vries, Amsterdam (NL)

(21) Appl. No.: 18/851,915

(22) PCT Filed: Mar. 30, 2023
(86) PCT No.: PCT/NL2023/050168

§ 371 Date: Sep. 27, 2024

(30) Foreign Application Priority Data
Mar. 30, 2022 (EP) 22165710.9

(51) Int. Cl. C12N 15/113; C12N 15/11

(52) U.S. Cl. CPC C12N 15/113; C12N 15/111; C12N 2310/14; C12N 2310/531; C12N 2320/34

(57) ABSTRACT

The invention relates to isolated antisense molecule capable of inhibiting the expression of the LMNA gene in a mammalian cell, wherein said antisense molecule comprises an anti-sense nucleic acid strand which is substantially complementary to a target region of a transcript encoded by the LMNA gene, wherein said antisense nucleic acid strand is at least complementary to SNP rs538089, rs505058 or rs4641 in said target region.

### FIELD

The invention relates to antisense molecules suitable for the treatment of heart diseases. More in particular, the invention relates to siRNAs and shRNAs suitable for use in the treatment of cardiac laminopathies, in particular dilated cardiomyopathy.

### BACKGROUND OF THE INVENTION

The currently used therapies for heart failure have successfully improved symptoms and survival for most forms of heart failure. However, in a minority of patients, these evidence-based treatments do not halt the progression of the disease, which leaves these patients with therapy-resistant heart failure. Ironically, this most often concerns young patients with heritable forms of heart failure, including the patients with heart failure caused by mutations in the lamin A/C (LMNA) gene.

Currently, more than 450 LMNA mutations have been reported, leading to a wide range of diseases (laminopathies) including dilated cardiomyopathy.

It is an object of the invention to provide a treatment for heritable forms of heart failure caused by mutations in the LMNA gene.

### SUMMARY OF THE INVENTION

The present invention provides an isolated antisense molecule capable of reducing or inhibiting expression of an allele of the LMNA gene in a mammalian cell, said antisense molecule comprises an antisense nucleic acid strand which is substantially complementary to a target region of a transcript encoded by the LMNA gene, wherein said antisense nucleic acid strand is at least complementary to SNP rs538089, rs505058 or rs4641 in said target region.

Preferably, said antisense molecule is capable of reducing expression of a mutant LMNA allele. In a most preferred embodiment, said antisense molecule is capable of reducing the expression of a mutant LMNA allele, while it leaves the unmutated LMNA allele unreduced.

Preferably, said isolated antisense molecule is selected from:
a. a double stranded nucleic acid (dsNA) or a chemically modified version thereof, or
b. an antisense oligonucleotide (ASO).

Preferably, said dsNA is RNA, more preferably a short hairpin (shRNA) or a small interfering RNA (siRNA). Preferably, said dsNA is dsRNA.

A preferred isolated antisense molecule according to the invention comprises an antisense nucleic acid strand of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region, provided that the antisense molecule specifically binds to a mutant LMNA allele. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides in said target region.

In a preferred embodiment of said isolated antisense molecule, said target region comprises the nucleic acid sequence selected from the group consisting of:

i.
(SEQ ID NO: 1)
GAUGACCUGCUCCAUCACCACCACGUGAGUGGUAGCCGCCGCUGA,
ii.
(SEQ ID NO: 2)
GAUGACCUGCUCCAUCACCACCAUGUGAGUGGUAGCCGCCGCUGA,
iii.
(SEQ ID NO: 3)
GAUGACCUGCUCCAUCACCACCACGGCUCCCACUGCAGCAGCUC,
and
iv.
(SEQ ID NO: 4)
GAUGACCUGCUCCAUCACCACCAUGGCUCCCACUGCAGCAGCUC

or a nucleic sequence being at least 80% homologous thereto. More preferably, at least 81, 82, 83, 84, 85, 86, 87, 88, 89, 90, 91, 92, 93, 94, 95, 96, 97, 98, 99% homologous thereto.

In another preferred embodiment, said target region comprises the nucleic acid sequence selected from the group consisting of:

i.
(SEQ ID NO: 5)
CCUGCUCCAUCACCACCACGUGAGUGGUAGCCGCCGC,
ii.
(SEQ ID NO: 6)
CCUGCUCCAUCACCACCAUGUGAGUGGUAGCCGCCGC,
iii.
(SEQ ID NO: 7)
CCUGCUCCAUCACCACCACGGCUCCCACUGCAGCAGC,
and
iv.
(SEQ ID NO: 8)
CCUGCUCCAUCACCACCAUGGCUCCCACUGCAGCAGC

or a nucleic sequence being at least 80% homologous thereto. More preferably, at least 81, 82, 83, 84, 85, 86, 87, 88, 89, 90, 91, 92, 93, 94, 95, 96, 97, 98, 99% homologous thereto.

In a preferred embodiment of the isolated antisense molecule according to the invention, said antisense nucleic acid strand comprises a nucleic acid sequence having a consecutive strand of at least 12, 13, 14, 15 16, 17, 18 or 19 nucleotides selected from the nucleic acid sequence according to SEQ ID NO:151, SEQ ID NO:152, SEQ ID NO: 153 or SEQ ID NO:154. Preferably, said antisense nucleic acid strand comprises a antisense strand as defined in Table 1 or a nucleic acid analogue sequence thereof. Preferably said antisense nucleic acid strand further comprises a sense strand as defined in Table 1 or a nucleic acid analogue sequence thereof. Preferably, said isolated antisense comprises the nucleic acid sequence selected from the group consisting of SEQ ID NO:71, SEQ ID NO: 72, SEQ ID NO:73 and SEQ ID NO:74.

In a preferred embodiment of said isolated dsNA, the dsNA comprises a sense nucleic acid strand of 15-30 nucleotides in length; and wherein the sense nucleic acid strand is complementary to said antisense nucleic acid strand and the sense nucleic acid strand and antisense nucleic acid strand form a duplex region.

Preferably, the antisense strand and the sense strand are operably linked by means of a loop strand to form a hairpin structure comprising a duplex structure and a loop structure. The loop structure may contain from 4 to 13 nucleotides, such as 4, 5 or 6 nucleotides. Preferably said loop contains the sequence UCAAGAC.

In a preferred embodiment of said isolated double stranded nucleic acid (dsNA) or an antisense oligonucleotide (ASO), said antisense nucleic acid strand is preferably 15-30 nucleotides in length. Preferably, said dsNA is a shRNA containing an isolated antisense strand of RNA of 15 to 30 nucleotides, preferably 19 nucleotides in length having a 5′ end and a 3′ end, wherein the antisense strand is preferably complementary to at least 15 nucleotides of said target region, and wherein preferably the 5′ end of the sense strand of RNA is operably linked to a G nucleotide to form a first segment of RNA, and an isolated antisense strand of RNA of 15 to 30 nucleotides in length having a 5′ end and a 3′ end, wherein preferably at least 12 nucleotides of the antisense and sense strands are complementary to each other and preferably form a small interfering RNA (siRNA) duplex under physiological conditions. Preferably said siRNA silences only one allele of the LMNA gene in the cell. The duplex formed by the two strands of RNA may be between 15 and 25 base pairs in length, preferably 19 base pairs in length. The antisense strand preferably is 19 nucleotides in length. The sense strand preferably is 19 nucleotides in length.

In the present invention, the sense and antisense strand of RNA may be operably linked together by means of an RNA loop strand to form a hairpin structure to form a “duplex structure” and a “loop structure.” These loop structures may be from 4 to 13, more preferably between 4-10 nucleotides in length. For example, the loop structure may be 4, 5 or 6 nucleotides long. The shRNA may further contain an overhang region. Such an overhang may be a 3′ overhang region or a 5′ overhang region. The overhang region may be, for example, from 1 to 6 nucleotides in length. In a preferred embodiment the 5′overhang has the nucleic acid sequence CCGG. In a preferred embodiment the 3′overhang has the nucleic acid sequence G.

The invention further provides a nucleic acid encoding the isolated double stranded nucleic acid or ASO of the invention. In a preferred embodiment, said antisense RNA of the invention, such as shRNA, is encoded by a oligonucleotide having the following structure: 5′-CCGGAA-19 bp sense strand-TCAAGAC-19 bp antisense strand-TTTTTTTG-3′ and reverse 5′-AATTCAAAAAAA-19 bp sense strand-GTCTTGA-19 bp antisense strand-TT-3′. Herein the sense strand is exactly the targeted sequence in the mRNA and the antisense strand its reverse complementary sequence that will eventually bind the mRNA and induce its breakdown and/or inhibition.

The present invention also provides an expression cassette comprising a nucleic acid encoding said antisense nucleic acid strand according to the invention. In a preferred embodiment, said nucleic acid encoding said antisense nucleic acid strand of the invention comprises the nucleic acid sequence according to any of SEQ ID Nos 75-150.

These expression cassettes may further contain a promoter. Such promoters can be regulatable promoters or constitutive promoters. Examples of suitable promoters include a CMV, RSV, pol II or pol III promoter. The expression cassette may further contain a polyadenylation signal, such as a synthetic minimal polyadenylation signal. The expression cassette may further contain a marker gene. In an embodiment, said expression cassette comprises 2 nucleic acids encoding said antisense nucleic acid strand according to the invention, wherein a first nucleic acid encodes an antisense strand which reduces the Lamin A isoform and a second nucleic acid encodes an antisense strand which is capable of reducing the Lamin C isoform. Expression of the antisense nucleic acid strands preferably is regulatable such that only one of the antisense nucleic acid strands is or can be expressed in a target cell.

The present invention provides a vector containing the expression cassette described above. Further, the vector may contain two expression cassettes, a first expression cassette containing a nucleic acid encoding the antisense strand of the RNA duplex and a second expression cassette containing the nucleic acid encoding a sense strand of the RNA duplex. Examples of appropriate vectors include adenoviral, lentiviral, adeno-associated viral (AAV), poliovirus, HSV, or murine Maloney-based viral vectors. In a preferred embodiment, the vector is a lentiviral vector. In another preferred embodiment, the vector is an AAV. Preferably said expression cassette encodes any of the ASOs or dsNA as described above.

The invention further provides a cell containing an expression cassette according to the invention. Preferably said cell is a mammalian cell, preferably a human cell. Preferably, said cell is a heart cell, preferably a Human induced pluripotent stem cell-derived cardiomyocyte (hiPSC-CM).

The invention further provides a pharmaceutical composition for reducing the expression of the LMNA gene in an organism, comprising the antisense molecule of the invention and a pharmaceutically acceptable carrier.

The invention further provides an isolated antisense molecule according to the invention for use in a medical treatment.

The invention further provides the isolated antisense molecule according to the invention, the nucleic acid encoding the isolated antisense molecule of the invention, the expression cassette of the invention, or the expression vector of the invention, the pharmaceutical composition according to the invention for use in the treatment of a heart failure. Preferably, said treatment is a treatment of a laminopathy, preferably dilated cardiomyopathy.

The invention further provides a method for selecting an antisense molecule which is suitable for the medical treatment of a disease caused by an autosomal dominant mutation present on a mutant allele of a gene, said method comprising:
a. Providing a nucleic acid sample of a subject suffering from a disease which is caused by an autosomal dominant mutation present on a mutant allele of a gene,
b. Determining the heterozygosity of the mutant allele of said gene which is responsible for said disease,
c. Determining the presence of an heterozygous SNP in said gene,
d. Determining the nucleic acid sequence of a target region comprising the heterozygous SNP of the mutant allele of said gene,
e. Selecting an isolated antisense molecule which is complementary to said target nucleic acid strand.

In a preferred embodiment, said antisense molecule is as described herein. Preferably, said gene is the LMNA gene. Preferably, said SNP is selected from rs538089, rs505058 and rs4641.

Said disease is a heart failure, specifically a laminopathy, such as dilated cardiomyopathy.

The invention further provides an in vitro method for reducing the expression of a mutant LMNA allele in a cell, comprising the following steps:
(a) introducing into the cell an antisense molecule of the invention which, upon introduction into a cell expressing a LMNA mutant allele, reduces the expression of the LMNA mutant allele; and
(b) maintaining the cell produced in step (a) for a time sufficient to obtain degradation of the mRNA transcript of the LMNA mutant allele, thereby reducing expression of the LMNA mutant allele in the cell. Preferably, said expression of the LMNA mutant gene is by at least 20%.

### BRIEF DESCRIPTION OF THE FIGURES

FIG. 1 shows an overview of the designed and tested siRNAs. The black outlined rectangle shows the common shared exon 10 between the lamin A and lamin C isoform. After SNP rs4641 (nucleotide in bold, C nucleotide=C allele; T nucleotide=T allele), lamin C continues with exon 10B (grey hatched rectangle) whereas lamin A continues with exon 11 (grey filled rectangle) and exon 12 (not shown). shRNAs were designed for each specific isoform and to target either the C allele or T allele.

FIG. 2 shows an overview of the results of the shRNAs targeting lamin C in the LMNA H222P hiPSC-CMs. FIG. 2A shows the relative expression of the C allele, T allele and total lamin A after treatment with the shRNAs targeting the C allele of rs4641 in lamin C. FIG. 2B shows the relative expression of the C allele, T allele and total lamin C after treatment with the shRNAs targeting the C allele of rs4641 in lamin C.

FIG. 2C shows the relative expression of the C allele, T allele and total lamin A after treatment with the shRNAs targeting the T allele of rs4641 in lamin C. FIG. 2D shows the relative expression of the C allele, T allele and total lamin C after treatment with the shRNAs targeting the T allele of rs4641 in lamin C. Bars present the mean and the errors bars present SEM. N=4-12 in 3-4 experiments.

FIG. 3 shows an overview of the results of the shRNAs targeting the C allele of rs4641 in lamin A in LMNA H222P and healthy hiPSC-CMs. FIG. 3A shows the relative expression of the C allele, T allele and total lamin A after treatment with the shRNAs targeting the C allele of rs4641 in lamin A in LMNA H222P hiPSC-CMs. FIG. 3B shows the relative expression of the C allele, T allele and total lamin C after treatment with the shRNAs targeting the C allele of rs4641 in lamin A in LMNA H222P hiPSC-CMs. FIG. 3C shows the relative expression of the C allele, T allele and total lamin A after treatment with the shRNAs targeting the C allele of rs4641 in lamin A in healthy hiPSC-CMs. FIG. 3D shows the relative expression of the C allele, T allele and total lamin C after treatment with the shRNAs targeting the C allele of rs4641 in lamin A in healthy hiPSC-CMs. Bars present the mean and the errors bars present SEM. N=4-9 in 2-3 experiments.

FIG. 4 shows an overview of the results of the shRNAs targeting the T allele of rs4641 in lamin A in LMNA H222P and healthy hiPSC-CMs. FIG. 4A shows the relative expression of the C allele, T allele and total lamin A after treatment with the shRNAs targeting the T allele of rs4641 in lamin A in LMNA H222P hiPSC-CMs. FIG. 4B shows the relative expression of the C allele, T allele and total lamin C after treatment with the shRNAs targeting the T allele of rs4641 in lamin A in LMNA H222P hiPSC-CMs. FIG. 4C shows the relative expression of the C allele, T allele and total lamin A after treatment with the shRNAs targeting the T allele of rs4641 in lamin A in healthy hiPSC-CMs. FIG. 4D shows the relative expression of the C allele, T allele and total lamin C after treatment with the shRNAs targeting the T allele of rs641 in lamin A in healthy hiPSC-CMs. Bars present the mean and the errors bars present SEM. N=5-9 in 2-3 experiments.

FIG. 5 shows the improvement of nuclear roundness after downregulation of the mutant LMNA allele by targeting the T allele of rs4641 by shRNA T17 in LMNA H222P hiPSC-CMs. Treatment of H222P hiPSC-CMs with a negative control shRNA (NC), the allele-specific shRNA C17 that downregulates the wildtype LMNA allele by targeting the C allele of rs4641 (C17) and the allele-specific shRNA T17 that downregulates the mutant LMNA allele by targeting the T allele of rs4641 (T17). N=305-364 from 3 different differentiations. Black bar indicates mean, ***=p<0.001.

FIG. 6: Schematic representation of the targeting siRNAs. The LMNA gene encodes 2 mRNA splice isoforms, LMNA and LMNC. SNP rs4641 (indicated in bold) is the last nucleotide shared by both isoforms in the middle of exon 10, after which LMNC is not splice and ends at exon 10, while LMNA splices immediately after rs4641 to exon 11. Because of the splicing we need to design LMNA (lower panel of siRNA) and LMNC (upper panel of siRNAs) specific siRNAs of which the sequences we will test for allele-specificity are indicated in the figure. Note that the siRNA sequences are written from 3′ to 5′, while all other sequences are written from 5′ to 3′.

FIG. 7. shRNA design and its conversion to active siRNA. The shRNA encoded by the virus consists of 2 complementary sequences (grey shaded) with a non-complementary sequence in the middle. These sequences, when they are transcribed from the virus, form a hairpin like structure, where complementary parts bind and the non-complementary part form a loop. This loop is removed by the enzyme Dicer to form an siRNA duplex. Of this duplex the so-called guide or antisense strand (dark grey shaded) is incorporated into the RNA induced silencing complex to bind and degrade the targeting mRNA, while the other passenger or sense strand of the siRNA duplex is degraded.

FIG. 8. Effects of shRNA targeting the C allele of LMNA on allele-specific expression of the LMNA and LMNC splice isoform respectively. A & C allele specific expression of the wildtype (C) allele and the mutant (T) allele of LMNA and LMNC respectively and the effect on total LMNA expression levels relatively to the expression in the SCR negative control condition. B & D show the percentage of expression of the mutant (T) and wildtype allele (C) of LMNA and LMNC respectively to visualize the balance between expression of the two alleles.

FIG. 9. Schematic representation of the mismatches in shRNA-C17 and shRNA-T17 targeting the LMNA splice isoform. Indicated in the figure are the original sequences and the shRNAs with the respective extra mismatches (indicated bold underscored). Note that the siRNA sequences are written from 3′ to 5′, while all other sequences are written from 5′ to 3′.

FIG. 10. Effects of additional mismatches in shC17 targeting the C allele of LMNA. A & C: allele specific expression of the wildtype (C) allele and the mutant (T) allele of LMNA and LMNC, respectively, and the effect on total LMNA/C expression levels relatively to the expression in the SCR negative control condition. B & D show the percentage of expression of the mutant (T) and wildtype allele (C) of LMNA and LMNC, respectively.

FIG. 11. Effects of shRNA targeting the T allele of LMNA on allele-specific expression of the LMNA and LMNC splice isoform respectively. A & C allele specific expression of the wildtype (C) allele and the mutant (T) allele of LMNA and LMNC respectively and the effect on total LMNA expression levels relatively to the expression in the SCR negative control condition. B & D show the percentage of expression of the mutant (T) and wildtype allele (C) of LMNA and LMNC respectively to visualize the balance between expression of the two alleles.

FIG. 12. Effects of additional mismatches in shT17 targeting the T allele of LMNA. A & C allele specific expression of the wildtype (C) allele and the mutant (T) allele of LMNA and LMNC respectively and the effect on total LMNA/C expression levels relatively to the expression in the SCR negative control condition. B & D show the percentage of expression of the mutant (T) and wildtype allele (C) of LMNA and LMNC respectively to visualize the balance between expression of the two alleles.

FIG. 13. Effects of shRNA targeting the C allele of LMNC on allele-specific expression of the LMNC and LMNA splice isoform respectively. A & C allele specific expression of the wildtype (C) allele and the mutant (T) allele of LMNC and LMNA respectively and the effect on total LMNA/C expression levels relatively to the expression in the SCR negative control condition. B & D show the percentage of expression of the mutant (T) and wildtype allele (C) of LMNC and LMNA respectively to visualize the balance between expression of the two alleles.

FIG. 14. Schematic representation of the mismatches in shRNA-C11, shRNA-C13 and shRNA-T14 targeting the LMNC splice isoform. Indicated in the figure are the original sequences and the shRNAs with the respective extra mismatches (indicated bold underscored). Note that the siRNA sequences are written from 3′ to 5′, while all other sequences are written from 5′ to 3′.

FIG. 15. Effects of additional mismatches in shC11 targeting the C allele of LMNC. A & C allele specific expression of the wildtype (C) allele and the mutant (T) allele of LMNC and LMNA respectively and the effect on total LMNA/C expression levels relatively to the expression in the SCR negative control condition. B & D show the percentage of expression of the mutant (T) and wildtype allele (C) of LMNC and LMNA respectively to visualize the balance between expression of the two alleles.

FIG. 16. Effects of additional mismatches in shC13 targeting the C allele of LMNC. A & C allele specific expression of the wildtype (C) allele and the mutant (T) allele of LMNC and LMNA respectively and the effect on total LMNA/C expression levels relatively to the expression in the SCR negative control condition. B & D show the percentage of expression of the mutant (T) and wildtype allele (C) of LMNC and LMNA respectively to visualize the balance between expression of the two alleles.

FIG. 17. Effects of shRNA targeting the T allele of LMNC on allele-specific expression of the LMNC and LMNA splice isoform respectively. A & C allele specific expression of the wildtype (C) allele and the mutant (T) allele of LMNC and LMNA respectively and the effect on total LMNA/C expression levels relatively to the expression in the SCR negative control condition. B & D show the percentage of expression of the mutant (T) and wildtype allele (C) of LMNC and LMNA respectively to visualize the balance between expression of the two alleles.

FIG. 18. Effects of additional mismatches in shT14 targeting the T allele of LMNC. A & C allele specific expression of the wildtype (C) allele and the mutant (T) allele of LMNC and LMNA respectively and the effect on total LMNA/C expression levels relatively to the expression in the SCR negative control condition. B & D show the percentage of expression of the mutant (T) and wildtype allele (C) of LMNC and LMNA respectively to visualize the balance between expression of the two alleles.

### DETAILED DESCRIPTION OF THE INVENTION

The present invention is based on the surprising finding of allele-specific silencing of the LMNA gene through expression of small interfering RNA (siRNA). The inventors have markedly diminished expression of a specific allele of this gene by targeting a region of the gene containing single-nucleotide polymorphism rs4641. The inventors sequenced the PCR amplicon containing rs4641 by using genomic DNA derived from a healthy control and a LMNA H222P human induced pluripotent stem cell (hiPSC) line and found that both hiPSC lines are heterozygous carriers of SNP rs4641 (C/T) and do not carry other variants.

SNP rs4641 is the last shared nucleotide of the two mRNA isoforms derived from LMNA, namely lamin A and lamin C. Both isoforms share the first 566 amino acids, but due to alternative splicing differ after exon 10. Lamin A continues with exons 11 and 12, whereas lamin C continues with exon 10B. Since SNP rs4641 is the last shared nucleotide of both isoforms, the inventors designed isoform specific siRNAs. Furthermore, the LMNA mutation, i.e. the disease-causing mutation, can be located on both alleles of the SNP (C or T allele), meaning that the inventors designed siRNAs for both SNP alleles on both the lamin A and lamin C isoform.

The inventors showed an improvement of nuclear roundness after downregulation of a mutant allele of LMNA by a shRNA targeting the region described above which demonstrates that this silencing is effective.

One goal of this invention is to provide a treatment wherein a mutant allele is suppressed. To discriminate between the mutant allele and wildtype (=healthy) allele, the inventors make use of a common variant (SNP rs4641) in LMNA. Therefore, in order to determine which allele needs to be suppressed, there is a need to determine on which allele the mutation is located. For example, if a certain mutation is located on the SNP T allele (i.e. the mutation and SNP T are in cis), the SNP T allele needs to be suppressed. However, if a mutation is located on the SNP C allele (i.e. the mutation and SNP C are in cis), SNP C allele needs to be suppressed. Since the inventors suppress the mutant allele independently of the specific mutation that causes the laminopathy, the isolated antisense molecule of the invention works for all mutations in the LMNA gene.

The inventors used a cell line harboring the LMNA H222P mutation. The H222P mutation is located on the rs4641 T allele. So, by suppressing the rs4641 C allele they would potentially aggravate the disease (as the wildtype is the healthy allele), whereas by suppressing the rs4641 T allele they would potentially alleviate the disease (suppressing mutant allele).

### I. Definitions

The term “antisense molecule” refers to an oligonucleotide molecule that contains sequence complementarity to target RNA molecules, such as mRNA, viral RNA, or other RNA species, and that reduces the function of their target RNA after sequence-specific binding. Examples of an antisense molecule are provided by the terms antisense oligonucleotide (ASO), siRNA, shRNA, interfering RNA molecule, and antisense oligomer, which terms may be used interchangeably herein below.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991) Nucleic Acids Research 19:5081; Ohtsuka et al., (1985) J Biol Chem 260:2605-2608; Rossolini et al., (1994) Mol Cell Probes 8:91-98).

A “nucleic acid fragment” is a portion of a given nucleic acid molecule.

The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

The term “nucleic acid analogue” refers to compounds which are analogous (structurally similar) to naturally occurring RNA and DNA. For example, RNA is a naturally occurring nucleic acid analogue of DNA. Nucleic acids are chains of nucleotides, which are composed of three parts: a phosphate backbone, a pentose sugar, either ribose or deoxyribose, and one of four nucleobases. An analogue may have any of these altered. Typically the analogue nucleobases confer, among other things, different base pairing and base stacking properties. Examples include universal bases, which can pair with all four canonical bases, and phosphate-sugar backbone analogues such as PNA. Artificial nucleic acids include peptide nucleic acid (PNA), Morpholino and locked nucleic acid (LNA), as well as glycol nucleic acid (GNA), threose nucleic acid (TNA) and hexitol nucleic acids (HNA). Each of these is distinguished from naturally occurring DNA or RNA by changes to the backbone of the molecule.

The term “nucleic acid analogue sequence of a nucleic acid sequence of a certain SEQ ID NO” refers to an nucleic acid sequence which is analogue to said SEQ ID NO. For instance SEQ ID NO: 83 refers to DNA sequence ccggaaatca ccaccacgtg agtggtcaag acccactcac gtggtggtga ttttttttg. The RNA analogue nucleic acid thereof has the Thymidine exchanged for Uracil. Another example of a nucleic acid analogue sequence of a nucleic acid sequence of SEQ ID NO 83 is a PNA nucleic acid sequence having the same bases.

The term “chemically modified version of a nucleic acid” refers to a chemical modification of one or more nucleotides in a nucleic acid which does not deteriorate the binding of said nucleic acid to the its complementary strand. A lengthy description of many known chemical modifications are set out in published PCT patent application WO 200370918, which is hereby incorporated by reference and will not be repeated here. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates).

The antisense molecules or dsNA molecules of the invention can be comprised of naturally occurring nucleotides or can be comprised of at least one modified nucleotide, such as a 2′-O-methyl modified nucleotide, a nucleotide comprising a 5′-phosphorothioate group, and a terminal nucleotide linked to a cholesteryl derivative or dodecanoic acid bisdecylamide group. Alternatively, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule containing less than 31 sequential nucleotides that is transcribed into an siRNA. Such an isolated siRNA may, for example, form a hairpin structure with a duplex 21 base pairs in length that is complementary or hybridizes to a sequence in a gene of interest, and remains stably bound under stringent. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. Please note that the terms “polypeptide” and “protein” are used interchangeably. Both terms refer to a molecule composed of a chain of amino acid residues bound together via covalent peptide bonds.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell (2001) “Molecular Cloning”. CSHL Press: Long Island, NY, USA.

A “homologous” DNA or RNA sequence is a sequence that is naturally associated with a host cell into which it is introduced.

A “vector” is defined to include, inter alia, any viral vector, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense molecule according to the invention, an antisense RNA, a nontranslated RNA in the sense or antisense direction, or a siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of an regulatable promoter that initiates transcription only when the host cell is exposed to some particular stimulus. In the case of a multicellular organism, the promoter can also be specific to a particular tissue or organ or stage of development.

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allele-specific” gene silencing refers to the specific silencing of one allele of a gene.

“RNA interference (RNAi)” is the process of sequence-specific, post-transcriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, siRNA induces degradation of target mRNA with consequent sequence-specific reduction or even inhibition of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

A “RNA duplex” or “dsRNA” refers to the structure formed by the complementary pairing between two regions of a RNA molecule. siRNA is “targeted” to a gene in that the nucleotide sequence of the duplex portion of the siRNA is complementary to a nucleotide sequence of the LMNA gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang of 0, 1, 2, 3, 4 or 5 nucleotides in length.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence.

As used herein, the term “region of complementarity” refers to the region on the antisense strand that is substantially complementary to a sequence, for example a target sequence, as defined herein.

The term “sense strand,” as used herein, refers to the strand of a dsRNA that includes a region that is substantially complementary to a region of the antisense strand.

“Treating” as used herein refers to ameliorating at least one symptom of, curing and/or preventing the development of a disease or a condition.

As used herein the term “encoded by” means that the DNA sequence in a gene or the SEQ ID NO is transcribed into the RNA of interest.

The term “capable of reducing the expression of the LMNA gene” refers to a process of substantial silencing of the LMNA gene through RNA interference. RNA interference can be triggered an antisense molecule, including by a double stranded nucleic acid (dsNA) or by a single stranded antisense oligonucleotide (ASO).

The “Lamin A/C” gene is also known as the LMNA gene. The LMNA gene encodes mRNA that is spliced into several alternative mRNAs which encode different lamin proteins, such as Lamin A or Lamin C.

Said process of substantial silencing of the LMNA gene through RNA interference preferably is directed at specifically silencing an allele of the LMNA gene, while expression of the second allele is not altered by said process. In a preferred embodiment one allele of the LMNA gene is silenced. An advantage thereof is that this enables silencing the mutant allele which alleviates the disease phenotype of many heritable forms of heart failure. Currently, more than 450 LMNA mutations have been reported, leading to a wide range of diseases (laminopathies) including dilated cardiomyopathy. Although no mutational hotspot(s) could be identified for all these laminopathies, they do share that the majority of these mutations have a dominant negative effect, indicating that the mutated gene product causes a phenotype in the presence of the wild type product.

The term “mutant LMNA allele” or “mutant LMNA gene” as used herein refers to a variant of the LMNA allele or gene of which the product has a negative effect on nuclear structure and function. Preferably, the mutant LMNA allele has a dominant negative effect, meaning that a heterozygotic expression of the mutant allele has a negative effect on nuclear structure and function.

The terms “antisense oligomer” or “antisense compound” or “antisense oligonucleotide” or “oligonucleotide” are used interchangeably and refer to a sequence of cyclic subunits (i.e. purine or pyrimidine), each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. The cyclic subunits may be based on ribose or another pentose sugar or, in certain embodiments, a morpholino group. Also contemplated are peptide nucleic acids (PNAs), locked nucleic acids (LNAs), and 2′-O-Methyl oligonucleotides, and other antisense agents known in the art.

Such an antisense oligomer can be designed to block or inhibit translation of mRNA or to inhibit natural pre-mRNA splice processing, or induce degradation of targeted mRNAs, and may be said to be “directed to” or “targeted against” a target sequence with which it hybridizes. In certain embodiments, the target sequence is a region surrounding or including an AUG start codon of an mRNA, a 3′ or 5′ splice site of a pre-processed mRNA, or a branch point. The target sequence may be within an exon or within an intron or a combination. The target sequence for a splice site may include an mRNA sequence having its 5′ end 1 to about 25 base pairs downstream of a normal splice acceptor junction in a preprocessed mRNA. A preferred target sequence for a splice is any region of a preprocessed mRNA that includes a splice site or is contained entirely within an exon coding sequence or spans a splice acceptor or donor site. Said preferred target sequence in a target region of a gene comprises a single nucleotide polymorphism (SNP), allowing degradation of a specific allele that, for example, carries a mutation. An oligomer is more generally said to be “targeted against” a biologically relevant target such as, in the present invention, a human LMNA gene pre-mRNA encoding the lamin A protein, when it is targeted against the nucleic acid of the target in the manner described above. Said antisense oligomer is preferably designed to induce degradation of a specific allele of the human LMNA gene, such as a SNP rs4641 T or SNP rs4641 C allele that carries a disease-causing mutation in cis configuration. As used herein “antisense oligonucleotides” (ASOs) mean agents that are unmodified or chemically modified single-stranded nucleic acid molecules (usually 15-30 nt in length), which can selectively hybridize to their target complementary sequence within mRNA through Watson-Crick base pairing. Formation of an ASO-mRNA heteroduplexes induces the effects as follows: 1) activates RNase H endonuclease or as in bacteria endoribonucleases—RNase III and RNase E-leading to degradation of the bound mRNA, and leaving the ASO intact; 2) causes translational arrest by steric hindrance of ribosomal activity; 3) inhibits mRNA splicing; 4) destabilizes pre-mRNA. Indeed, what effect will occur depends on the ASO chemical composition and location of hybridization, but the subsequent result is specific down-regulation of the target gene and protein expression.

As used herein the term “rs4641” refers to an SNP located on chr1:156137743 (GRCh38.p13). See world wide web at ncbi.nlm.nih.gov/snp/rs4641 released Apr. 9, 2021. As used herein, it also refers to the corresponding nucleotide polymorphism when it is present in the transcript as encoded by the LMNA gene.

As used herein, the term “LMNA mutation” refers to a mutation in the LMNA gene which preferably has a dominant negative effect and specifically causes a laminopathy, more specifically cardiomyopathy.

Disclosed herein is a strategy that results in substantial silencing of a specific targeted allele via RNA interference. Use of this strategy results in markedly diminished in vitro and in vivo expression of targeted genes or alleles. This strategy is useful in reducing expression of targeted genes or alleles in order to model biological processes or to provide therapy for human diseases.

As used herein the term “substantial silencing” in the context of silencing an allele means that the mRNA of the targeted allele is specifically inhibited and/or degraded by the presence of the introduced antisense molecule, such that expression of the targeted allele is reduced by about 10% to 100% as compared to the level of expression seen when the antisense molecule of the invention is not present. Generally, when an allele is substantially silenced, it will have at least 40%, 50%, 60%, 70%, e.g., 71%, 72%, 73%, 74%, 75%, 76%, 77%, 78%, 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the isolated antisense molecule is not present. In contrast, the expression of the non-targeted allele is not substantially altered as compared to the level of expression seen when the antisense molecule of the invention is not present. As used herein the term “substantial silencing” in the context of silencing a gene means that the mRNA of the targeted allele is inhibited and/or degraded by the presence of the introduced isolated antisense molecule, such that expression of the targeted allele is reduced by about 10% to 100% as compared to the level of expression seen when the isolated antisense molecule is not present. Generally, when an allele is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the isolated antisense molecule is not present. As used herein the term “substantially normal activity” means the level of expression of an allele or gene when an isolated antisense molecule has not been introduced to a cell.

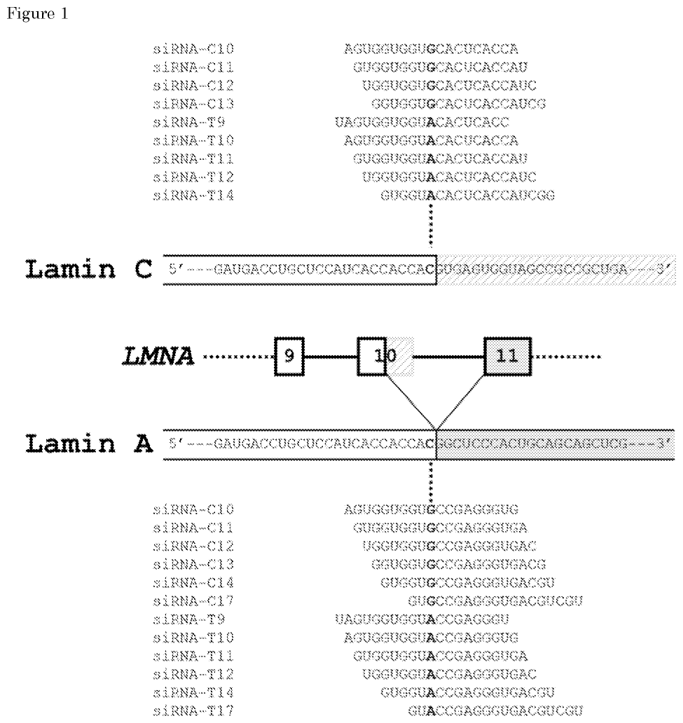

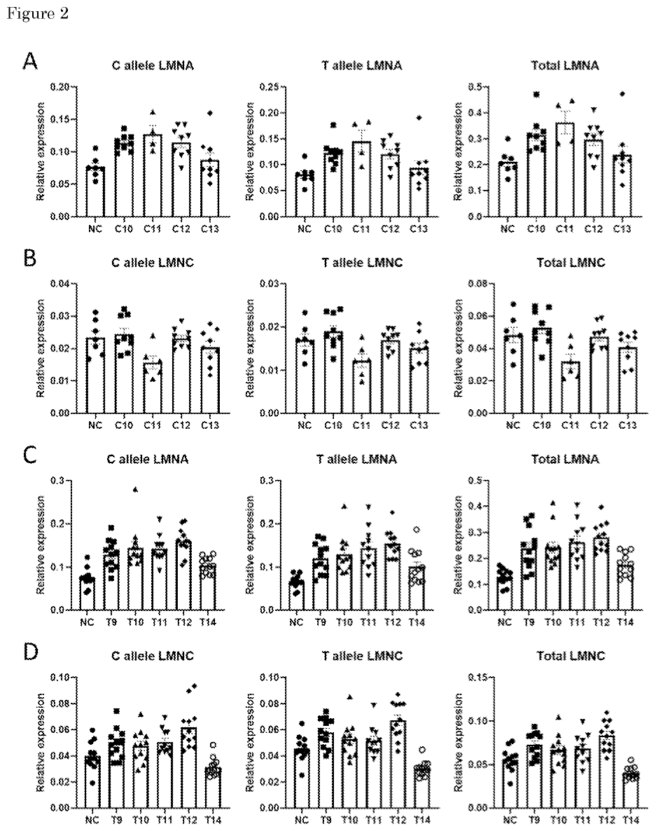

As outlined in the strategy in FIG. 1, the inventors developed siRNA that would specifically eliminate production of the Lamin A/C protein from the mutant allele, i.e. the allele having the disease-causing mutation. By exploiting base pair differences between wild type and mutant alleles, the inventors successfully allele-specifically silenced expression of the mutant LMNA mRNA without interfering with expression of the wild type mRNA. Because lamin A/C is an essential protein it is critically important that efforts be made to silence only the mutant allele. This allele-specific strategy has obvious therapeutic potential for the treatment of diseases caused by LMNA mutations, in particular laminopathies, including dilated cardiomyopathy.

In a preferred embodiment, said antisense nucleic acid strand is of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region. Preferably, there are no more than 2 mismatches, more preferably no more than 1 mismatch. If present, said mismatch will not interfere with the allele-specific targeting of, for example, SNP rs538089, rs505058 or rs4641. In certain preferred embodiments there is no mismatch in complementarity. Suitably, a skilled person can design mismatch positions with the highest discrimination potential depending on the type of nucleotide changes as described by Huang et al. [Nucleic Acids Res 2009, 37, 7560-7569, doi: 10.1093/nar/gkp835]. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides.

In a preferred embodiment, the isolated antisense molecule is selected from:
a. a double stranded nucleic acid (dsNA) or a chemically modified version thereof, or
b. a single stranded antisense oligonucleotide (ASO).

Preferred embodiments of dsNA are synthetic double stranded small interfering RNA (siRNA) and vector driven short hairpin RNA (shRNA). Both siRNA and vector driven shRNA have been demonstrated to be effective in in vitro and in vivo applications, each with their respective advantages. Most siRNA are structurally designed to promote efficient incorporation into the Ago2 containing RISC, the RNase III containing Dicer-substrate design improves the efficiency of siRNA at least 10-fold by initial association and processing at the pre-RISC. Vector driven shRNA utilizes the host microRNA biogenesis pathway, which appears to be very efficient. siRNA is more readily chemically modified while shRNA expression can be modulated and regulated by specific promoters. In addition, allele-specific suppression by shRNA may be long lasting, as a vector encoding the shRNA may be integrated into the genome of a cell.

In an embodiment, to accomplish intracellular expression of the therapeutic siRNA, preferably an RNA molecule is constructed containing two complementary strands or a hairpin sequence (such as a 21-bp hairpin) representing sequences directed against the target region encoded a part of the LMNA gene comprising SNP rs538089, rs505058 or rs4641. The siRNA, or a nucleic acid encoding the siRNA, is introduced to the target cell, such as a diseased heart cell. The siRNA reduces target mRNA and protein expression of the targeted, mutated allele.

In an embodiment, the dsRNA of the invention is chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, NY, USA, which is hereby incorporated herein by reference. Chemical modifications may include, but are not limited to 2′ modifications, introduction of non-natural bases, covalent attachment to a ligand, replacement of phosphate linkages with thiophosphate linkages, and combinations thereof. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues, and combinations thereof. Preferably, the chemical groups that can be used to modify the dsRNA include, without limitation, methylene blue; bifunctional groups, preferably bis-(2-chloroethyl)amine; N-acetyl-N′-(p-glyoxylbenzoyl) cystamine; 4-thiouracil; and psoralen. In one preferred embodiment, the linker is a hexaethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The ends of the dsRNA is preferably formed by a triple-helix structure.

The construct encoding the therapeutic siRNA is configured such that the one or more strands of the siRNA are encoded by a nucleic acid that is immediately contiguous to a promoter and that are under control of said promoter meaning that said promoter will drive expression of said nuclei acid after introduction into a target cell. The construct is introduced into the target cell, such as by injection, allowing for diminished expression of the target-allele in the cell.

The present invention provides an expression cassette comprising a nucleic acid encoding at least said antisense nucleic acid strand. In some embodiments, said expression cassette comprises a nucleic acid encoding the complete isolated antisense molecule according to the invention.

In an embodiment, the siRNA of the invention may form a hairpin structure that contains a duplex structure and a loop structure. The loop structure may contain from 4 to 13, more preferably 4-10 nucleotides, such as 4, 5 or 6 nucleotides. Preferably said loop contains the sequence UCAAGAC. The duplex is less than 30 nucleotides in length, such as from 19 to 25 nucleotides. The siRNA may further contain an overhang region. Such an overhang may be a 3′ overhang region or a 5′ overhang region. The overhang region may be, for example, from 1 to 6 nucleotides in length.

The present invention also provides an expression cassette containing an isolated nucleic acid sequence encoding a first segment, a second segment located immediately 3′ of the first segment, and a third segment located immediately 3′ of the second segment, wherein the first and third segments are each less than 30 base pairs in length and each more than 10 base pairs in length, and wherein the sequence of the third segment is the complement of the sequence of the first segment, and wherein the isolated nucleic acid sequence functions as a small interfering RNA molecule (siRNA) targeted against a gene of interest. The expression cassette may be contained in a vector, such as a viral vector.

The expression cassette may further contain a pol II promoter, as described herein. Examples of pol II promoters include regulatable promoters and constitutive promoters. For example, the promoter may be a CMV or RSV promoter. The expression cassette may further contain a polyadenylation signal, such as a synthetic minimal polyadenylation signal. The nucleic acid sequence may further contain a marker gene. The expression cassette may be contained in a viral vector. An appropriate viral vector for use in the present invention may be an adenoviral, lentiviral, adeno-associated viral (AAV), poliovirus, herpes simplex virus (HSV) or murine Maloney-based viral vector.

The present invention provides a method of reducing the expression of a specific gene product in a cell by contacting a cell with an expression cassette described above. It also provides a method of treating a patient by administering to the patient a composition of the expression cassette described above.

The present invention further provides a method of reducing the expression of a specific gene product in a cell by contacting a cell with an expression cassette as described above.

The present invention also provides a method of treating a patient, by administering to the patient a composition containing an expression cassette according to the invention. Said expression cassette preferably encompasses shC17, shC17 mm5, shC17 mm7, shC13 mm17, shT14, shT17, shT17 mm5 and shT17 mm7 as described in Example 2.

### II. Nucleic Acid Molecules of the Invention

Sources of nucleotide sequences from which the present nucleic acid molecules can be obtained include any vertebrate, preferably mammalian, cellular source. In addition to a DNA sequence encoding a siRNA, the nucleic acid molecules of the invention include single and double-stranded interfering RNA molecules, which are also useful to reduce expression of a target allele.

Oligonucleotide-mediated mutagenesis is a method for preparing substitution variants. This technique is known in the art. Briefly, nucleic acid encoding a siRNA can be altered by hybridizing an oligonucleotide encoding the desired mutation to a DNA template, where the template is the single-stranded form of a plasmid or bacteriophage containing the unaltered or native gene sequence. After hybridization, a DNA polymerase is used to synthesize an entire second complementary strand of the template that will thus incorporate the oligonucleotide primer, and will code for the selected alteration in the nucleic acid encoding siRNA. Generally, oligonucleotides of at least 25 nucleotides in length are used. An optimal oligonucleotide will have 12 to 15 nucleotides that are completely complementary to the template on either side of the nucleotide(s) coding for the selected alteration. This ensures that the oligonucleotide will hybridize properly to the single-stranded DNA template molecule. The oligonucleotides are readily synthesized using techniques known in the art.

The DNA template can be generated by those vectors that are either derived from bacteriophage M13 vectors (the commercially available M13 mp 18 and M13 mp 19 vectors are suitable), or those vectors that contain a single-stranded phage origin of replication. Thus, the DNA that is to be altered may be inserted into one of these vectors to generate single-stranded template.

Alternatively, single-stranded DNA template may be generated by denaturing double-stranded plasmid (or other) DNA using standard techniques.

For alteration of the native DNA sequence the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the altered form of the DNA, and the other strand (the original template) encodes the native, unaltered sequence of the DNA. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the altered DNA. The altered region is then removed and placed in an appropriate vector, generally an expression vector of the type typically employed for transformation of an appropriate host.

The method described immediately above may be modified such that a homoduplex molecule is created wherein both strands of the plasmid contain the alteration(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(*S) (which can be obtained from the Amersham Corporation). This mixture is added to the template-oligonucleotide complex. Upon addition of DNA polymerase to this mixture, a strand of DNA identical to the template except for the altered bases is generated. In addition, this new strand of DNA will contain dCTP-(*S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be altered. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

### III. Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the siRNA, or the like. Such elements may be included in the DNA as desired to obtain the optimal performance of the siRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein. For example, Sambrook and Russell, infra, provides suitable methods of construction.

The recombinant DNA can be readily introduced into the host cells, e.g., mammalian, bacterial, yeast or insect cells by transfection with an expression vector composed of DNA encoding the siRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described hereinbelow, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed above, a “transfected”, or “transduced” host cell or cell line is one in which the genome has been altered or augmented by the presence of at least one heterologous or recombinant nucleic acid sequence. The host cells of the present invention are typically produced by transfection with an antisense molecule, a DNA sequence in a plasmid expression vector, a viral expression vector, or as an isolated linear DNA sequence. The transfected DNA can become a chromosomally integrated recombinant DNA sequence, which is composed of sequence encoding the siRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell”, comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent expressing an antisense molecule, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent expressing an antisense molecule and the therapeutic agent is delivered in situ.

### VI. Delivery Vehicles for the Expression Cassettes of the Invention

The selection and optimization of a particular expression vector for expressing a specific siRNA in a cell can be accomplished by obtaining the nucleic acid sequence of the siRNA, possibly with one or more appropriate control regions (e.g., promoter, insertion sequence); preparing a vector construct comprising the vector into which is inserted the nucleic acid sequence encoding the siRNA; transfecting or transducing cultured cells in vitro with the vector construct; and determining whether the siRNA is present in the cultured cells.

Vectors for cell gene therapy include viruses, such as replication-deficient viruses. Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present invention. Such retroviruses further have utility for the efficient transduction of nucleic acid sequences into cells in vivo. Retroviruses have been used extensively for transferring nucleic acid material into cells.

An advantage of using retroviruses for gene therapy is that the viruses insert the nucleic acid sequence encoding the siRNA into the host cell genome, thereby permitting the nucleic acid sequence encoding the siRNA to be passed on to the progeny of the cell when it divides. Promoter sequences in the LTR region have been reported to enhance expression of an inserted coding sequence in a variety of cell types. Some disadvantages of using a retrovirus expression vector are (1) insertional mutagenesis, i.e., the insertion of the nucleic acid sequence encoding the siRNA into an undesirable position in the target cell genome which, for example, leads to unregulated cell growth and (2) the need for target cell proliferation in order for the nucleic acid sequence encoding the siRNA carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells. The adenovirus also has been used as an expression vector in muscle cells in vivo.

Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ. In addition, the ability of the adenovirus vector to accomplish in situ tumor transduction has allowed the development of a variety of anticancer gene therapy methods for non-disseminated disease. In these methods, vector containment favors tumor cell-specific transduction.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination results. The desired recombinants are identified by screening individual plaques generated in a lawn of complementation cells.

Most adenovirus vectors are based on the adenovirus type 5 (Ad5) backbone in which an expression cassette containing the nucleic acid sequence of interest has been introduced in place of the early region 1 (E1) or early region 3 (E3). Viruses in which E1 has been deleted are defective for replication and are propagated in human complementation cells (e.g., 293 or 911 cells), which supply the missing gene E1 and pIX in trans.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical, electroporation, scrape loading, microparticle bombardment or by cellular uptake as a chemical complex (e.g., calcium or strontium co-precipitation, complexation with lipid, complexation with ligand)). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

### VII. Methods for Selecting an Antisense Molecule

In diseases caused by an autosomal dominant mutation, one of the alleles of a gene contains a mutation which has a dominant negative effect. It is therefore desirable to inhibit such mutation present in the affected allele while allowing the unaffected allele of said gene to be expressed. In order to select a suitable antisense molecule which is capable of selectively inhibiting, i.e. reducing, the affected allele, a nucleic acid sample is provided from a patient suffering from a disease caused by an autosomal dominant mutation. Many such diseases are known. A non-limiting list of examples of such disease include tuberous sclerosis, breast cancer, neurofibromitosis Type I, Huntington disease, hypertrophic cardiomyopathy, polycystic kidney disease, osteogenesis imperfecta, chondrodysplasia, centronuclear myopathy and Marfan syndrome. In principle, any such disease may be treated by the antisense molecule selected according to the invention. The method of the invention requires a nucleic acid, preferably a DNA sample of the patient. Any biological sample from the patient which contains DNA may be used, including but not limited to a blood sample, saliva, cheek mucosa etc. In certain embodiments RNA may be used. DNA may be isolated from said sample according to methods known in the art. Subsequently, the presence of an autosomal dominant mutation is determined by determining the nucleic acid sequence of said gene of both alleles present. Subsequently, the alleles are screened for the presence of a heterozygous SNP in the alleles of said gene. In a preferred embodiment a heterozygous SNP is determined by selecting a synonymous SNP, as synonymous SNPs are expected to have the highest minor allele frequencies (MAF). Then the nucleic acid sequence of the sense strand of said SNP of said gene is determined using methods known in the art. Subsequently, an isolated antisense molecule is selected which comprises an antisense nucleic acid strand which is substantially complementary to a target region of a transcript encoded by said gene, wherein said antisense nucleic acid strand is at least complementary to said SNP in said target region.

In a preferred embodiment, said method of selecting an antisense molecule of the invention further comprises a step wherein the selected antisense molecule is tested in a cell to determine the effect on reduction of expression of said gene. Preferably said method comprises determining the effect of reduction of expression of the mutant and/or the unmutated allele of said gene. Preferably said method comprises a step of determining a change in allelic imbalance of said gene. Preferably, said step is performed on a cell from the patient.

In a preferred embodiment, the isolated antisense molecule is selected from:
a. a double stranded nucleic acid (dsNA) or a chemically modified version thereof, or
b. an antisense oligonucleotide (ASO).

Preferably, said dsNA is RNA, more preferably a short hairpin (shRNA) or a small interfering RNA (siRNA). Preferably, said dsNA is dsRNA.

The term siRNA, as is used herein, refers to a double stranded RNA molecule composed of 15-31 base pairs (bp), preferably 20-25 bp, and comprising two RNA molecules. The term shRNA, as is used herein, refers to a single RNA molecule that forms a hairpin structure comprising a partially double stranded RNA molecule composed of 15-31 bp, preferably 20-25 bp, and a single stranded loop.

In a preferred embodiment the isolated antisense molecule according to the invention, said antisense nucleic acid strand is of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides in said target region.

In a preferred embodiment of the isolated double stranded nucleic acid (dsNA) according to the invention, the dsNA comprises a sense nucleic acid strand of 15-30 nucleotides in length; and the sense nucleic acid strand is complementary to said antisense nucleic acid strand and the sense and antisense nucleic acid strands form a duplex region. Preferably, said dsNA is a shRNA containing an antisense nucleic acid strand of RNA of 15 to 30 nucleotides, preferably 19 nucleotides in length having a 5′ end and a 3′ end, wherein the antisense nucleic acid strand is preferably complementary to at least 15 nucleotides of said target region, and wherein preferably the 5′ end of the sense strand of RNA is operably linked to a G nucleotide to form a first segment of RNA, and a sense strand of RNA of 15 to 30 nucleotides in length having a 5′ end and a 3′ end, wherein preferably at least 12 nucleotides of the antisense and sense strands are complementary to each other and preferably form a small interfering RNA (siRNA) duplex under physiological conditions.

Preferably, the antisense strand and the sense strand are operably linked by means of an RNA loop strand to form a hairpin structure comprising a duplex structure and a loop structure.

Preferably, the loop structure preferably contains from 4 to 13, more preferably between 4 and 10 nucleotides. In an embodiment, said loop structure contains 4, 5 or 6 nucleotides. Preferably said loop contains the sequence UCAAGAC.

The duplex formed by the two strands of RNA may be between 15 and 25 base pairs in length, preferably 19 base pairs in length. The antisense strand preferably is 19 nucleotides in length. The sense strand preferably is 19 nucleotides in length. The shRNA may further contain an overhang region. Such an overhang may be a 3′ overhang region or a 5′ overhang region. The overhang region may be, for example, from 1 to 6 nucleotides in length.

The invention further provides a nucleic acid encoding the isolated antisense molecule of the invention. In a preferred embodiment, said shRNA of the invention is encoded by an oligonucleotide having the following structure: forward 5′-CCGGAA-19 bp sense strand-TCAAGAC-19 bp antisense strand-TTTTTTTG-3′ and/or reverse 5′-AATTCAAAAAAA-19 bp sense strand-GTCTTGA-19 bp antisense strand-TT-3′. Herein the sense strand has the same sequence as the targeted sequence in the mRNA and the antisense strand its reverse complementary sequence that will eventually bind the mRNA and induce its breakdown and/or inhibition.

Preferably, said gene is selected from the LMNA, Potassium Voltage-Gated Channel Subfamily Q Member 1 (KCNQ1), Neurofibromin 1 (NF1), Fibrillin 1 (FBN1), BReast CAncer Gene 1 (BRCA1), BRCA2, Tuberous Sclerosis Complex 1 (TSC1) and TSC2 gene. Preferably, said SNP is selected from rs1057128, rs8234 and rs17215465.

Preferably, said disease is a heart failure, tuberous sclerosis, breast cancer, neurofibromitosis Type I, Huntington disease, hypertrophic cardiomyopathy, polycystic kidney disease, osteogenesis imperfecta, chondrodysplasia, centronuclear myopathy and Marfan syndrome. Preferably said heart failure is a laminopathy, preferably dilated cardiomyopathy.

The invention will now be illustrated by the following non-limiting Examples.

### EXAMPLES

### Example 1

### Materials and Methods

Human iPSC Culture

The LMNA H222P SV1122 hiPSC line derived from a male patient carrying the LMNA H222P mutation was generated in Hamburg, Germany. The UN1-22 line was derived from a healthy male and the full characterization was previously published (Shinnawi, 2015 Stem Cell Reports 2015; 5:582-596). Both lines are heterozygous for SNP rs4641 located in LMNA. We cultured colonies of both hiPSCs lines in mTeSR-1 (StemCell Technologies; 85850) on plates coated with 1:500 diluted growth factor reduced Matrigel (Corning; FAL356231). We passaged the cells every 4-6 days via dissociation with 0.5 mM EDTA (Invitrogen; 15575-038), and replated them in mTeSR-1 supplemented with 2 μM Thiazovivin (Selleck Chemicals; S1459). Between passages we changed the mTeSR-1 medium every day, except for the first day after passaging.

Cardiac Differentiation of hiPSC

We performed cardiac differentiation following a previously published protocol with slight adaptations (Burridge, 2014 Nat Methods 2014; 11:855-860). We induced differentiation 3-5 days after passaging by changing to CDM3 medium (RPMI 1640, Gibco 21875; 500 μg/ml human serum albumin, Sigma A9731; 213 μg/ml L-ascorbic acid 2 phosphate, Sigma A8960; 1% penicillin/streptomycin) supplemented with 6 μM CHIR99021 (Stemgent; 04-0004-10) for two days, followed by CDM3 with 2 μM Wnt-C59 (Selleck Chemicals; S7037) for two days. From day 4 to day 10 we changed the medium every other day for RPMI/B27 medium (RPMI-1640, 2% B27 supplement minus insulin (Gibco; A1895601), 1% penicillin/streptomycin). Spontaneous contraction could be identified from day 8 onwards. From day 10 onwards we changed the medium on hiPSC-CM once every week with CDM3 medium without glucose (RPMI-1640 without glucose, Gibco; 11879) supplemented with 20 mM sodium-lactate (Sigma-Aldrich; L7022) dissolved in 1M HEPES-solution for at least 2 weeks to metabolically select the cardiomyocytes. After selection, we changed the medium once a week with CDM3 medium with glucose.

We dissociated purified populations of cardiomyocytes by TrypLE Express (Gibco; 12604) incubation for 15 minutes and plated them on matrigel coated plates in RPMI/B27 with 2 μM Thiazovivin for further experiments. We conducted all the experiments on hiPSC-CMs 40-60 days after start of differentiation and replicated each observation in 2 to 4 independent experiments with hiPSC-CMs from different differentiations.

### Plasmid Generation

For shRNA expression we used the pLKO.1 backbone with puromycin as selection marker (pLKO.1 puro; Addgene; 8453). For cloning of shRNA sequences we designed the following oligonucleotides: forward 5′-CCGGAA-19 bp sensestrand-TCAAGAC-19 bp antisense strand-TTTTTTTG-3′ and reverse 5′-AATTCAAAAAAA-19 bp sense strand-GTCTTGA-19 bp antisense strand-TT-3′. Where the sense strand is exactly the mRNA targeting sequence and the antisense strand its reverse complementary sequence that will eventually bind the mRNA. We designed the shRNA sequences according to Huang et al. (2014). We annealed 1 nmol of these oligonucleotides and cloned them into the AgeI and EcoRI restriction sites in the pLKO.1-puro plasmid. ShRNA sequences are detailed in Table 1. We verified all plasmid sequences by Sanger sequencing and excluded plasmids containing mutations.

### Virus Production

To produce third-generation lentivirus of pLKO.1-puro based constructs we co-transfected 4×10\hat{ }6 HEK293T cells with 4 μg of the expression plasmid, 2.7 μg pMDLg/pRRE, 1 μg pRSV-Rev, 1.4 μg pVSVG using Genejammer (Agilent; 204130) according to the manufacturer's protocol. The next day we changed the HEK293T medium to CDM3 medium. We collected this medium containing the produced lentivirus 24 hours after previous change and used it directly for hiPSC-CMs transduction.

### HiPSC-CM Infection

We dissociated hiPSC-CMs 2 to 4 days before lentiviral transduction to ensure homogenous cell populations between conditions. We freshly added 2 ml/well in a 6-well plate of medium with virus to the hiPSC-CMs. After 24 hours we removed the medium containing the virus and replaced it with fresh CDM3 medium. After 5 days we changed the medium and started the puromycin selection with 8 g/ml puromycin in CDM3 medium for 48 hours. We harvested the cells after the puromycin selection.

### RNA Isolation

We isolated total RNA from all samples using 1 ml TriReagent (Sigma Aldrich; T9424). We added TriReagent directly to live cells growing on a dish. We performed total RNA isolation according to the manufacturer's protocol.

Allele-Specific qRT-PCR for mRNA

To detect the mRNA expression of each LMNA allele, 250 ng to 1 μg RNA was DNAse treated with DNAseI amplification grade (Invitrogen; 18068015) and reverse transcribed using Superscript II reverse transcriptase (Invitrogen; 18064014) with oligo-dT and random hexamer primers according to the manufacturer's protocol. cDNA was diluted 5 times and 2 μl used as input for the qPCR.

qPCR was performed using 1 μM primers (Table 2) and LightCycler 480SYBR Green master 1 (Roche; 04887352001) on a LightCycler 480 system II (Roche) using the following cycling program: 5 minutes pre-incubation at 95° C.; 40 cycles of 10 seconds denaturation at 95° C., 20 seconds annealing (temperatures in Table 2), and 20 seconds elongation at 72° C. Data were analyzed using LinRegPCR quantitative PCR analysis software (Ruijter, 2009) and the starting concentration of transcripts estimated by this software was corrected for the geometric mean of the estimated starting concentration of the three reference genes GAPDH, HPRT and TBP.

TABLE 1
shRNA targeting sequences

| | shRNA | Location | Sense strand | Antisense strand |
|---|---|---|---|---|
| Target | number | mismatch | (5′-3′) | (5′-3′) |
| Negative | 175 | — | GAAATGTACTGCGTGGAGA | TCTCCACGCAGTACATTTC |
| control | | | SEQ ID NO: 9 | SEQ ID NO: 10 |
| C allele | 209 | C10 | TCACCACCACGTGAGTGGT | ACCACTCACGTGGTGGTGA |
| LMNC | | | SEQ ID NO: 11 | SEQ ID NO: 12 |
| | 210 | C11 | CACCACCACGTGAGTGGTA | TACCACTCACGTGGTGGTG |
| | | | SEQ ID NO: 13 | SEQ ID NO: 14 |
| | 211 | C12 | ACCACCACGTGAGTGGTAG | CTACCACTCACGTGGTGGT |
| | | | SEQ ID NO: 15 | SEQ ID NO: 16 |
| | 212 | C13 | CCACCACGTGAGTGGTAGC | GCTACCACTCACGTGGTGG |
| | | | SEQ ID NO: 17 | SEQ ID NO: 18 |
| T allele | 213 | T9 | ATCACCACCATGTGAGTGG | CCACTCACATGGTGGTGAT |
| LMNC | | | SEQ ID NO: 19 | SEQ ID NO: 20 |
| | 214 | T10 | TCACCACCATGTGAGTGGT | ACCACTCACATGGTGGTGA |
| | | | SEQ ID NO: 21 | SEQ ID NO: 22 |
| | 215 | T11 | CACCACCATGTGAGTGGTA | TACCACTCACATGGTGGTG |
| | | | SEQ ID NO: 23 | SEQ ID NO: 24 |
| | 216 | T12 | ACCACCATGTGAGTGGTAG | CTACCACTCACATGGTGGT |
| | | | SEQ ID NO: 25 | SEQ ID NO: 26 |
| | 217 | T14 | CACCATGTGAGTGGTAGCC | GGCTACCACTCACATGGTG |
| | | | SEQ ID NO: 27 | SEQ ID NO: 28 |
| C allele | 230 | C10 | TCACCACCACGGCTCCCAC | GTGGGAGCCGTGGTGGTGA |
| LMNA | | | SEQ ID NO: 29 | SEQ ID NO: 30 |
| | 231 | C11 | CACCACCACGGCTCCCACT | AGTGGGAGCCGTGGTGGTG |
| | | | SEQ ID NO: 31 | SEQ ID NO: 32 |
| | 232 | C12 | ACCACCACGGCTCCCACTG | CAGTGGGAGCCGTGGTGGT |
| | | | SEQ ID NO: 33 | SEQ ID NO: 34 |
| | 233 | C13 | CCACCACGGCTCCCACTGC | GCAGTGGGAGCCGTGGTGG |
| | | | SEQ ID NO: 35 | SEQ ID NO: 36 |
| | 234 | C14 | CACCACGGCTCCCACTGCA | TGCAGTGGGAGCCGTGGTG |
| | | | SEQ ID NO: 37 | SEQ ID NO: 38 |
| | 235 | C17 | CACGGCTCCCACTGCAGCA | TGCTGCAGTGGGAGCCGTG |
| | | | SEQ ID NO: 39 | SEQ ID NO: 40 |
| T allele | 236 | T9 | ATCACCACCATGGCTCCCA | TGGGAGCCATGGTGGTGAT |
| LMNA | | | SEQ ID NO: 41 | SEQ ID NO: 42 |
| | 237 | T10 | TCACCACCATGGCTCCCAC | GTGGGAGCCATGGTGGTGA |
| | | | SEQ ID NO: 43 | SEQ ID NO: 44 |
| | 238 | T11 | CACCACCATGGCTCCCACT | AGTGGGAGCCATGGTGGTG |
| | | | SEQ ID NO: 45 | SEQ ID NO: 46 |
| | 239 | T12 | ACCACCATGGCTCCCACTG | CAGTGGGAGCCATGGTGGT |
| | | | SEQ ID NO: 47 | SEQ ID NO: 48 |
| | 240 | T14 | CACCATGGCTCCCACTGCA | TGCAGTGGGAGCCATGGTG |
| | | | SEQ ID NO: 49 | SEQ ID NO: 50 |
| | 241 | T17 | CATGGCTCCCACTGCAGCA | TGCTGCAGTGGGAGCCATG |
| | | | SEQ ID NO: 51 | SEQ ID NO: 52 |

TABLE 2
Primer sequences used for qRT-PCR

| Target | Forward primer | Reverse primer | Annealing |
|---|---|---|---|
| (mRNA) | (5′-3′) | (5′-3′) | temperature |
| C allele | ACCTGCTCCATCACCACCAC | AGCCGCTGGCAGATGCCTTG | 66 |
| LMNA | SEQ ID NO: 53 | SEQ ID NO: 54 | |
| T allele | ACCTGCTCCATCACCACCAT | AGCCGCTGGCAGATGCCTTG | 66 |
| LMNA | SEQ ID NO: 55 | SEQ ID NO: 56 | |
| Total | ACCTGCTCCATCACCACC | AGCCGCTGGCAGATGCCTTG | 66 |
| LMNA | SEQ ID NO: 57 | SEQ ID NO: 58 | |
| C allele | ACCTGCTCCATCACCACCAC | TGGCCCCAGTGCAGGCTC | 66 |
| LMNC | SEQ ID NO: 59 | SEQ ID NO: 60 | |
| T allele | ACCTGCTCCATCACCACCAT | TGGCCCCAGTGCAGGCTC | 66 |
| LMNC | SEQ ID NO: 61 | SEQ ID NO: 62 | |
| Total | ACCTGCTCCATCACCACC | TGGCCCCAGTGCAGGCTC | 66 |
| LMNC | SEQ ID NO: 63 | SEQ ID NO: 64 | |
| GAPDH | ACCCACTCCTCCACCTTTGAC | ACCCTGTTGCTGTAGCCAAATT | 60 |
| | SEQ ID NO: 65 | SEQ ID NO: 66 | |
| HPRT | TGACACTGGCAAAACAATGCA | GGTCCTTTTCACCAGCAAGCT | 60 |
| | SEQ ID NO: 67 | SEQ ID NO: 68 | |
| TBP | GCTCACCCACCAACAATTTAG | TCTGCTCTGACTTTAGCACCTG | 60 |
| | SEQ ID NO: 69 | SEQ ID NO: 70 | |

### Results

To test our hypothesis of silencing a mutant allele by allele-specific siRNAs targeting a common variant independently of the causal mutation, we searched for common variants in LMNA in our two human induced pluripotent stem cell (hiPSC) lines. In LMNA 3 known common single-nucleotide polymorphisms (SNPs) are present, namely rs538089, rs505058 and rs4641 with a heterozygosity of 21.8%, 20.4% and 32.8% respectively. We sequenced 3 PCR amplicons containing these 3 SNPs by using genomic DNA derived from our hiPSC lines and found that both hiPSC lines are heterozygous carriers of SNP rs4641 (C/T) and do not carry the other variants. For this reason, we choose SNP rs4641 as the targetable variant in LMNA.

SNP rs4641 is the last shared nucleotide of the two mRNA isoforms derived from LMNA, namely lamin A and lamin C. Both isoforms share the first 566 amino acids, but due to alternative splicing differ after exon 10, lamin A continues with exons 11 and 12, whereas lamin C continues with exon 10B. Since SNP rs4641 is the last shared nucleotide of both isoforms, we needed to design isoform specific siRNAs. Furthermore, the LMNA mutation can be located on both alleles of the SNP (C or T allele), meaning that we need siRNAs for both SNP alleles on both the lamin A and lamin C isoforms. Based on literature and gained knowledge about the design of siRNAs, we designed constructs for each allele of each isoform (see FIG. 1). Since we envision to deliver our constructs via gene therapy using short hairpin RNAs (shRNAs), we used shRNAs in our experiments to test immediately the effects of the shRNAs in human induced pluripotent stem cell derived cardiomyocytes (hiPSC-CMs).

No Allele-Specific Effects Observed of the shRNAs to Downregulate Lamin C

First, we started to test our shRNAs targeting the C allele of lamin C in LMNA H222P hiPSC-CMs. As expected, we observed no downregulation of either the Cor T allele of the lamin A isoform, indicating that our designed shRNAs towards the C allele of lamin C are isoform specific (FIG. 2, panel A). We observed downregulation of the C allele of lamin C by shRNA C11 (FIG. 2, panel B). However, we also observed downregulation of the T allele of lamin C by this shRNA, indicating that this shRNA is not allele-specific. All other tested shRNAs did not show any downregulation (FIG. 2, panel B).

Next, we moved on to test our shRNAs targeting the T allele of lamin C in LMNA H222P hiPSC-CMs. Also these shRNAs are isoform specific since no downregulation of the lamin A isoforms are observed (FIG. 2, panel C). We observed downregulation of the T allele of lamin C by shRNA T14 (FIG. 2, panel D). However, this effect is not allele-specific since the same amount of downregulation is observed on the C allele of lamin C. All other tested shRNAs did not show any downregulation (FIG. 2, panel D).

To conclude, none of our designed shRNAs targeting either the C allele or the T allele showed allele-specific downregulation of lamin C.

shRNAs C17 and T17 Give Isoform and Allele-Specific Downregulation of Lamin A

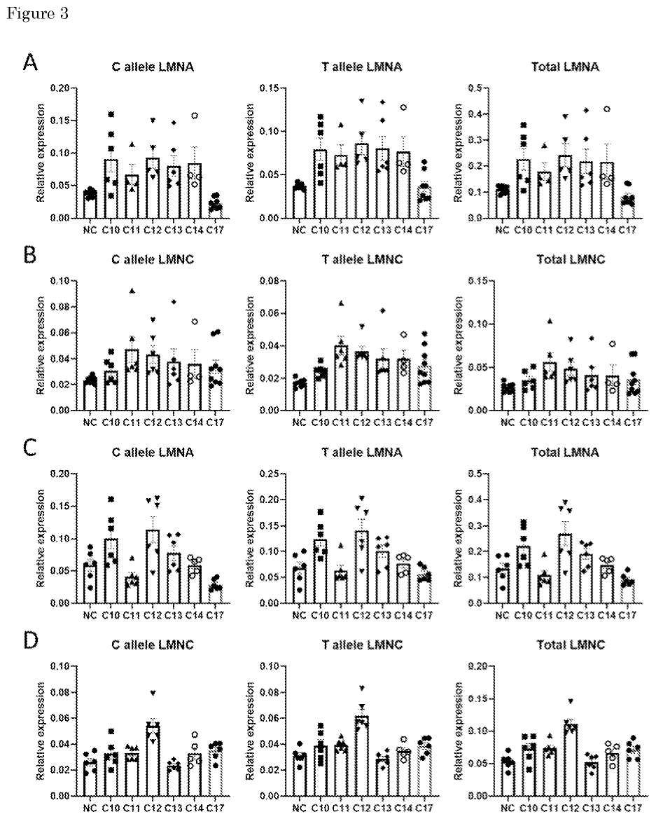

Next, we tested the shRNAs targeting the alleles of the lamin A isoform, starting with targeting the C allele of lamin A. We observed a downregulation by shRNA C17 of 40% of the targeted C allele of lamin A in LMNA H222P hiPSC-CMs (FIG. 3, panel A). Furthermore, this effect is allele-specific since we did not observe downregulation of the T allele of lamin A. Moreover, the allele-specific effect is also isoform specific, since shRNA C17 showed no downregulation of any lamin C allele (FIG. 3, panel B). All other tested shRNAs showed no lamin A nor lamin C downregulation. Subsequently, we tested the same shRNAs in healthy hiPSC-CMs. We observed the same effects of shRNA C17 with now even a higher downregulation of 50% of the targeted C allele of lamin A (FIG. 3, panel C and D). However, we now also observed a slight effect on the T allele of lamin A.

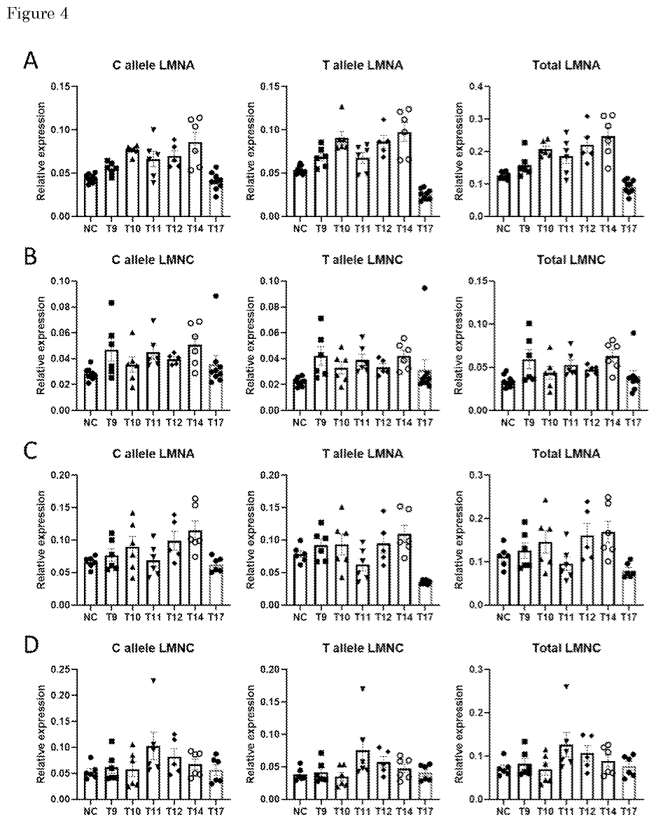

Lastly, we tested the shRNAs targeting the T allele of lamin A. We observed a downregulation of 53% obtained by shRNA T17 in LMNA H222P hiPSC-CMs (FIG. 4, panel A). More importantly, this observed effect is allele-specific and isoform specific (FIG. 4, panel B). The other tested shRNAs showed no downregulation. As before, we tested the same shRNAs in healthy hiPSC-CMs. Similarly, shRNA T17 showed an isoform and allele-specific downregulation of 55% (FIG. 4, panel C and D).

To conclude, we found 2 shRNAs which downregulate isoform and allele-specifically either the C or T allele of SNP rs4641 of lamin A with an efficiency around 50% in 2 different hiPSC-CMs lines.

Downregulation of the Mutant H222P Allele with shRNA T17 Improves Nuclear Roundness

To assess the effect of either downregulation of the C allele or T allele by shRNAs C17 and T17 on the nuclear phenotype, we treated H222P hiPSC-CMs with a negative control shRNA, shRNA C17 (downregulation of the C allele) and shRNA T17 (downregulation of the T allele). Plasmid cloning of LMNA PCR products surrounding both the SNP and the mutation on cDNA of the H222P-line and Sanger sequencing of bacterial clones revealed that the heterozygous H222P mutation is located on the same allele as the T allele of rs4641. Therefore, downregulation of the rs4641 C allele in H222P hiPSC-CMs results in downregulation of the wildtype allele, which is expected to aggravate the phenotype, whereas downregulation of the rs4641 T allele would downregulate the mutant allele and is expected to alleviate the phenotype.

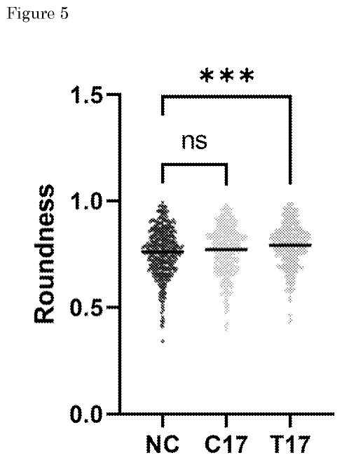

Since LMNA encodes the nuclear lamina, which provides support to the nuclear membrane, LMNA is important for the nuclear architecture. Therefore, the nuclear shape is a measure of how severely a cell is affected by a certain mutation. We assessed the effect of shRNAs-based downregulation of either the wildtype or mutant allele on the nuclear roundness of the cells. We measured the nuclear roundness after treatment with the 2 different conditions compared to the negative control. We did not observe a decrease of nuclear roundness after downregulation of the wildtype allele. However, therapeutically much more interesting, downregulation of the rs4641 T allele and thereby the mutant allele improved the nuclear roundness significantly (p<0.001). See FIG. 5. Thus, downregulation of the T allele and thereby downregulating the H222P mutant allele by shRNA T17 results in an improvement of the nuclear roundness in LMNA H222P hiPSC-CMs, which indicates that these LMNA H222P hiPSC-CMs are less severely affected by the mutation after downregulation of the mutant allele by shRNA-targeting of SNP rs4641.

### Example 2

### Materials and Methods

See Example 1 with the following additions:

Cardiac differentiation of hiPSC was performed as described, or by following a CHIR-dilution protocol, consisting of 6 microM CHIR99021 in 3 ml CDM3 medium at day 0, addition of 2 ml CDM3 medium at day 1, addition of another 1 ml CDM3 medium at day 2, and 2 microM WNT-C59 in CDM3 on day 3. From day 4 or 5 (no dilution vs dilution protocol) until day 10, we changed the medium every other day for RPMI/B27 medium (RPMI-1640, 2% B27 supplement minus insulin (Gibco; A1895601), 1% penicillin/streptomycin).

Cardiomyocytes were dissociated by TrypLE Express (Gibco; 12604) incubation for 15 to 45 minutes, after which they were plated on Matrigel-coated plates in RPMI/B27 with 2 μM Thiazovivin for further experiments.

For plasmid generation, we designed shRNA sequences according to Huang et al. (2014) and added mismatches based on the known interaction regions of miRNAs with their targets and on the results of the original shRNA selection. Additional shRNA targeting sequences and primer sequences used for qRT-PCR are included in Tables 3 and 4, respectively.

### Results

### SNP Identification for Allele-Specific Targeting

To allow allele-specific downregulation of LMNA by targeting common SNPs, we searched the coding region of LMNA for synonymous SNPs, because these are expected to have the highest minor allele frequencies (MAF). Consequently, the highest number of patients will be heterozygous for these SNPs, which allows allele-specific targeting. This search revealed 3 synonymous SNPs (rs538089, rs505058, and rs4641) in LMNA with a MAF in the Genome Aggregation Database (gnomAD) population of 18, 24 and 21% respectively. These MAFs indicate that according to the Hardy-Weinberg Equilibrium 29% of patients should be heterozygous for SNP rs538089, 36% heterozygous for rs505058, and 33% for SNP rs4641. This means that targeting these respective SNPs would allow to treat a similar amount of the LMNA patients, independent of the disease causing mutation. We screened 8 LMNA mutation carriers from the Amsterdam UMC outpatient clinic and a hiPSC-line with the H222P mutation in LMNA for heterozygosity for these SNPs, which revealed 5 patients and the hiPSC-line to be heterozygous for rs4641 and no heterozygosity for rs538089 or rs505058. Therefore, we focused our allele-specific shRNA approach at SNP rs4641.

Design of Allele-Specific shRNAs Against Both Alleles of Rs4641 in LMNA and LMNC

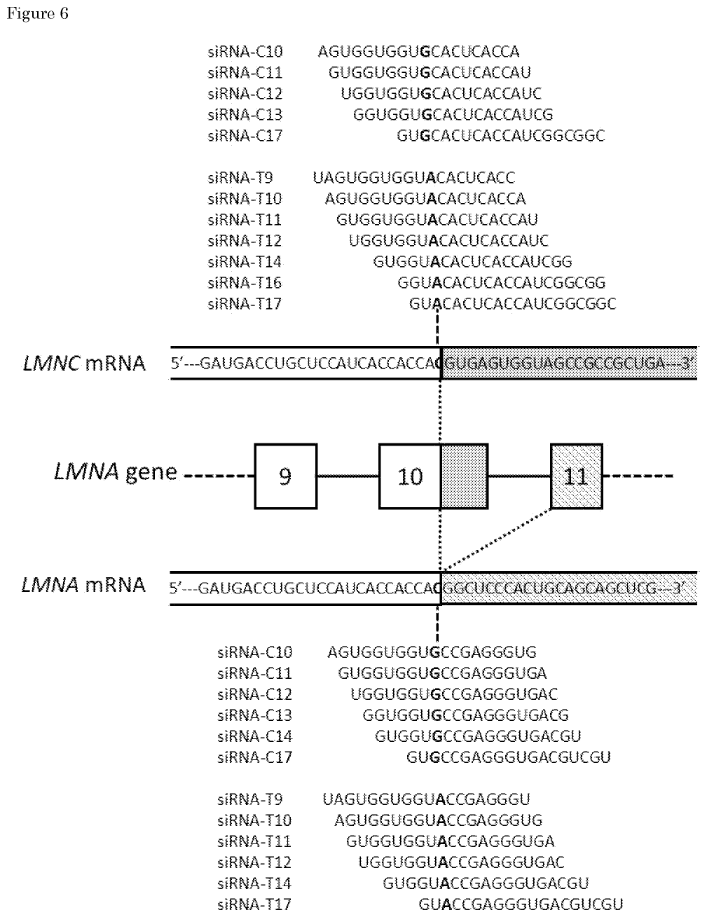

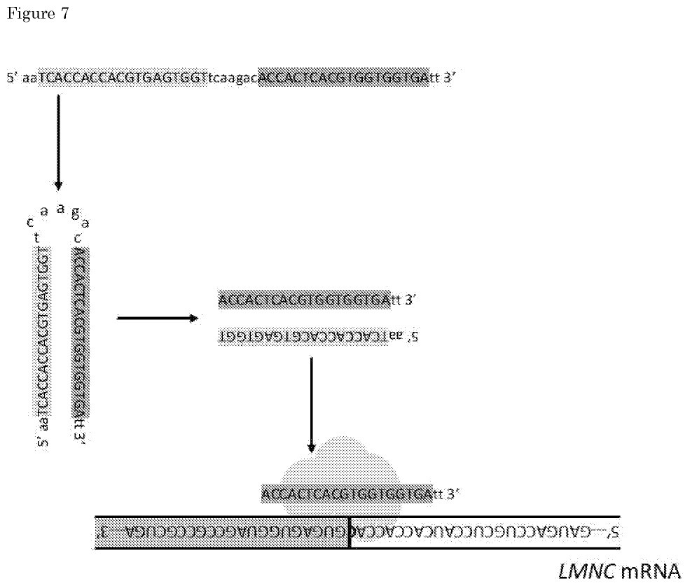

The LMNA gene encodes for two mRNA splice isoforms, LMNA and LMNC, which share the sequence encoding the first 566 amino acids. Alternative splicing in exon 10 leads to differences between the two isoforms in their C-terminus, where the LMNC isoform is not spliced and ends at the end of exon 10, while the LMNA isoform uses a splice site in the middle of exon 10 to splice towards exon 11 and 12 (FIGS. 1 and 6). SNP rs4641 is located at the last nucleotide that is shared between the two isoforms. This, in combination with the data of Huang et al., (Huang et al., 2009. Nucleic Acids Res 37:7560-7569), who showed that the highest allele-specificity is obtained when the nucleotide that distinguishes both alleles is located in the middle of the siRNA, indicates that we need to design allele-specific siRNAs targeting both LMNA and LMNC separately. Furthermore, because both the T allele and C allele of the SNP can reside on the mutant allele in different patients, we do need to design siRNAs targeting both alleles of SNP rs4641. Based on the data of Huang et al., we selected 6 siRNAs predicted to target the C allele of LMNA with a mismatch to the non-targeted T allele at positions 10, 11, 12, 13, 14, and 17; and 6 siRNAs predicted to target the T allele of LMNA with a mismatch to the non-targeted C allele at positions 9, 10, 11, 12, 14, and 17 (FIG. 6). Using a similar approach we selected 5 siRNAs predicted to target the C allele of LMNC with a mismatch to the non-targeted T allele at positions 10, 11, 12, 13, and 17; and 7 siRNAs predicted to target the T allele of LMNC with a mismatch to the non-targeted C allele at positions 9, 10, 11, 12, 14, 16, and 17 (FIG. 6). Because we envision therapeutic delivery of these siRNAs via viral mediated shRNA expression, we also expressed these siRNAs as viral shRNA constructs in vitro according to the design depicted in FIG. 7.

### Allele-Specific Targeting of Rs4641 in the LMNA Isoform

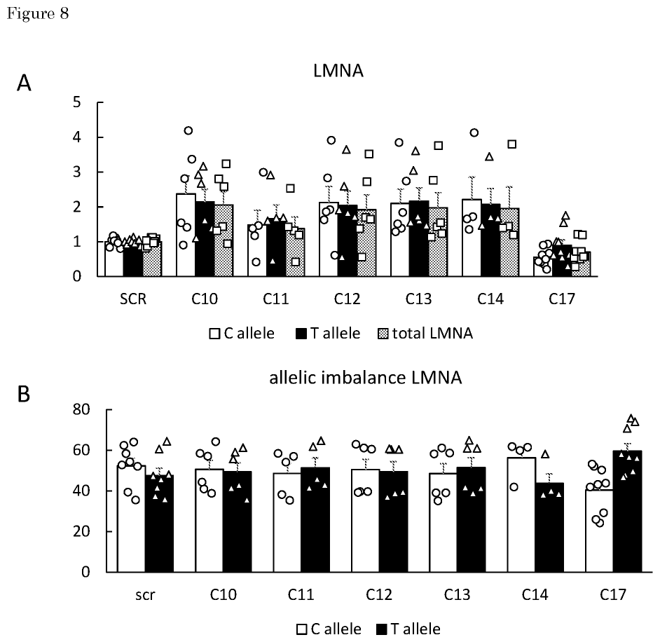

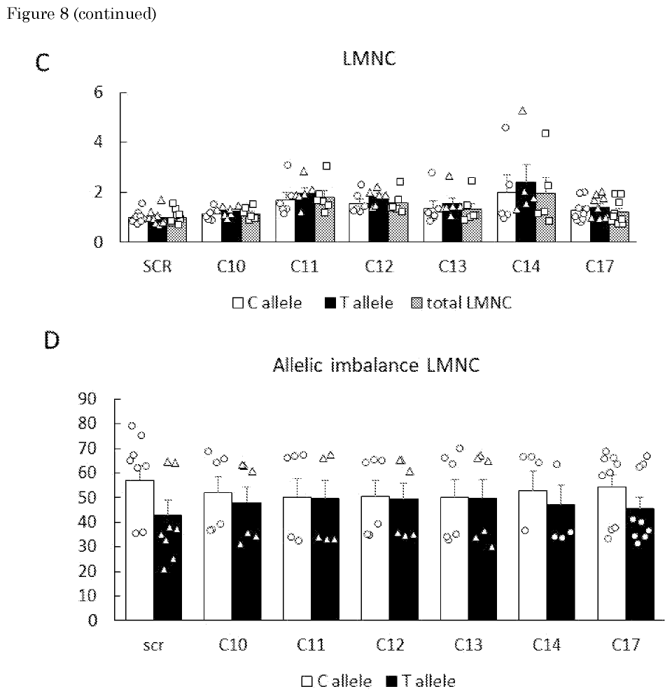

To test the shRNAs, we lentivirally transduced cardiomyocytes derived from the H222P hiPSC-line. This line is derived from a male patient with an Emery-Dreifuss-Muscular-Dystrophy phenotype, which affects both skeletal and cardiac muscle (Bonne et al., 2000. Ann Neurol 48:170-80). After lentiviral shRNA transduction we compared by allele-specific qRT-PCR expression levels of mutant and wildtype LMNA alleles to the expression levels in cells transduced with a scrambled negative control shRNA (Scr). Targeting the C allele of rs4641 in the LMNA splice isoform, which resides in this H222P hiPSC-line on the wildtype LMNA allele, revealed one shRNA, shC17, which downregulated the wildtype LMNA allele by almost 50% and the non-targeted mutant allele by only 10% (FIG. 8A). Because most mutations in LMNA, including the H222P mutation, have a dominant-negative mode of action, which means that the mutated allele interferes with the function of the wildtype allele, the allelic balance between a wildtype and such a mutated allele determines the disease severity. Therefore, we also investigated to what extent the targeting of the C allele of rs4641 affected the allelic balance between both LMNA alleles (FIG. 8B). This revealed in this experiment in the SCR negative control cells an almost equal expression of both alleles (52:48%), which in the cells transduced by shC17 shifted to a relative under-expression of the targeted wildtype LMNA allele (40:60%). The other shRNAs to our surprise did not downregulate any LMNA allele, but instead resulted in an upregulation of both wildtype and mutant LMNA alleles. Although we are only targeting the LMNA splice isoform by these shRNAs we also performed allele-specific qRT-PCR on LMNC, which revealed no downregulation of any LMNC allele and thus also no strong effects on the balance between the mutant and wildtype LMNC isoforms (FIG. 8C/D).

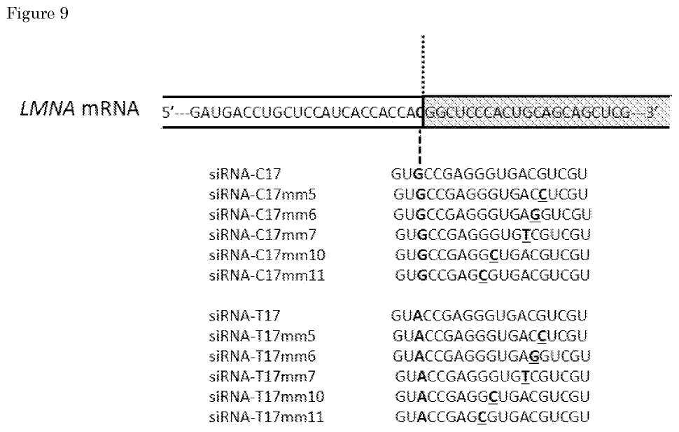

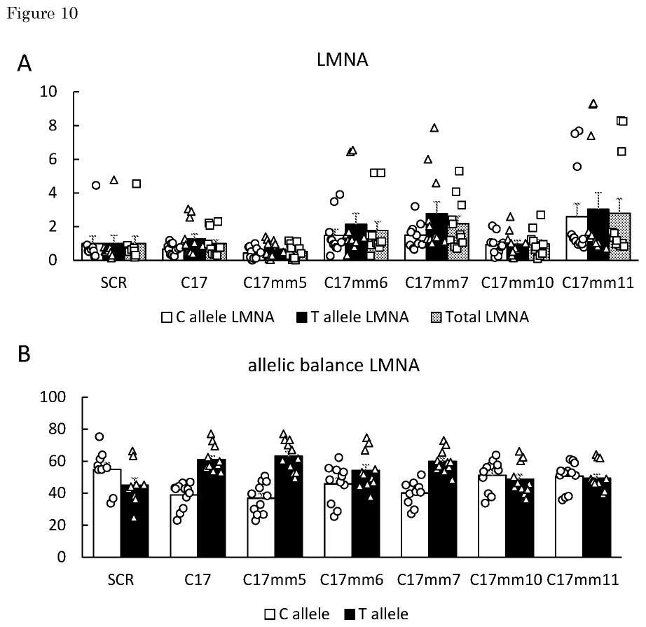

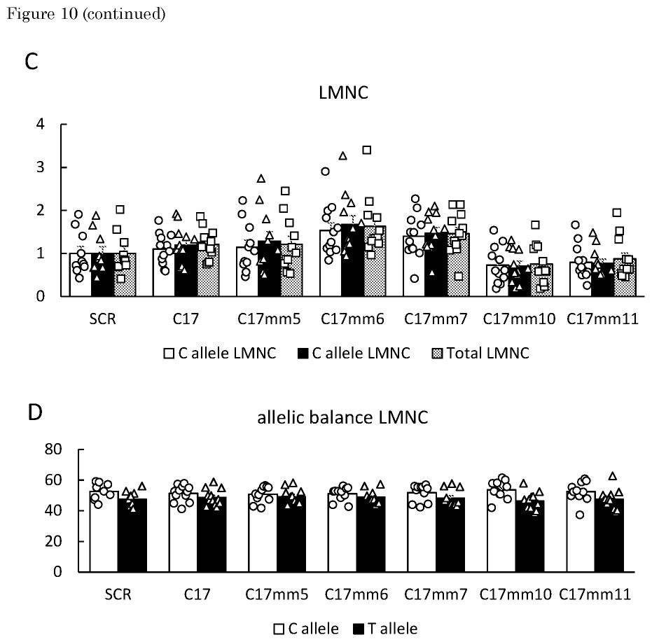

Previous studies suggested that introduction of additional mismatches in the seed region or the Argonaute recognition region of shRNA can improve discrimination between targeted and non-targeted alleles. Because we had a 10% knockdown of the non-targeted T allele of rs4641 of the LMNA splice isoform by the shC17 for LMNA, we added extra mismatches to this shC17 sequence in an attempt to further reduce this targeting. We added mismatches at position 5, 6, and 7 in the seed region and 10, and 11 in the argonaute recognition region (FIG. 9). We changed the nucleotides in the shRNA in such a way that the GC content was stable, however, we expect other changes to have similar effects. Allele-specific qRT-PCR after transduction of the original shC17 sequences and the shRNAs with additional mismatches revealed that shC17 mm5 more strongly induced a downregulation of the targeted C allele of rs4641 in the LMNA isoform, but also a stronger downregulation of the non-targeted T allele (FIG. 10A). This resulted in a slightly stronger shift in allelic balance compared to the original C17 sequence (36:63% shC17 mm5 and 39:61% shC17; FIG. 10B). Interestingly, shC17 mm7 resulted in a comparable allelic imbalance of LMNA (40:60%), which was induced by a stronger upregulation of the non-targeted allele than the targeted allele. We again showed that these shRNAs targeting the LMNA splice isoform did not affect the LMNC splice isoform levels. To further study whether the absolute levels of the mutant allele or the allelic imbalance is the most important factor that determines disease severity in patients and phenotype in our hiPSC-CMs we decided to further study shC17, shC17 mm5 and shC17 mm7 and compare their effect on the disease phenotype in hiPSC-CMs.

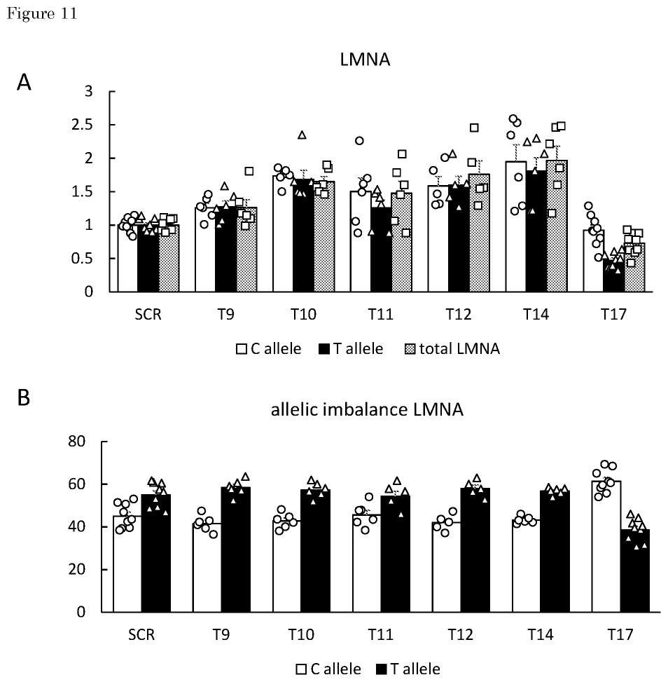

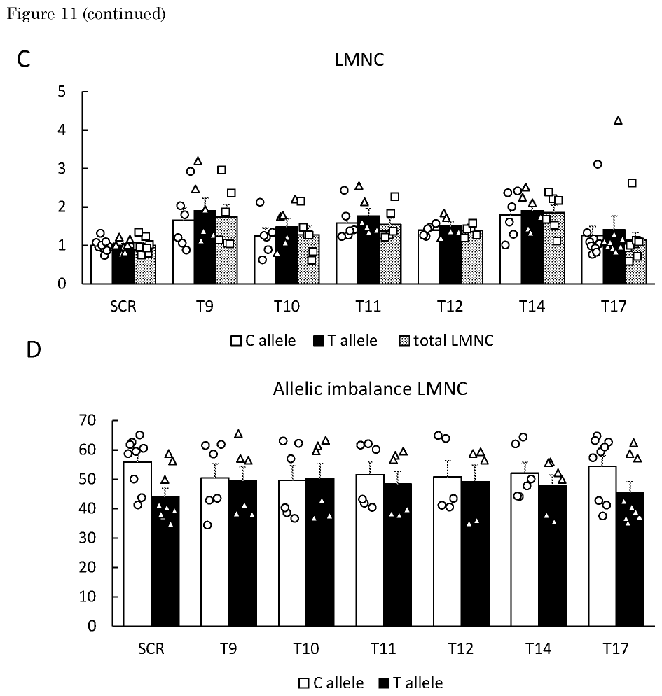

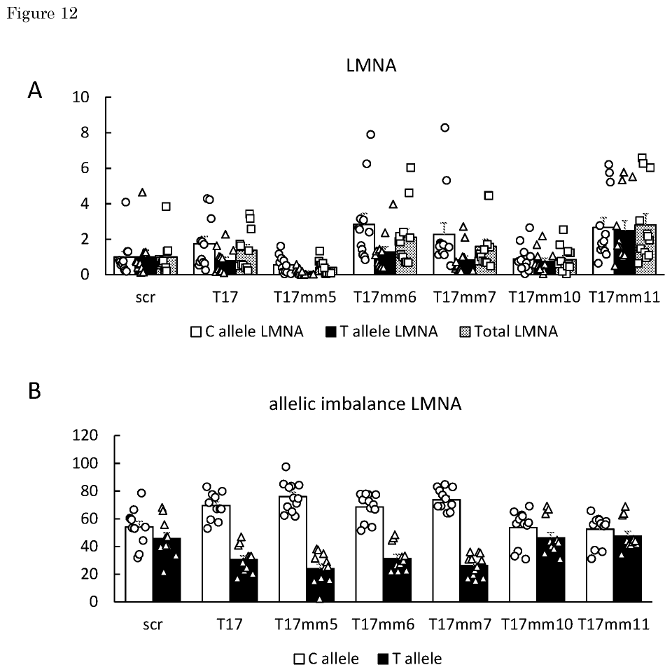

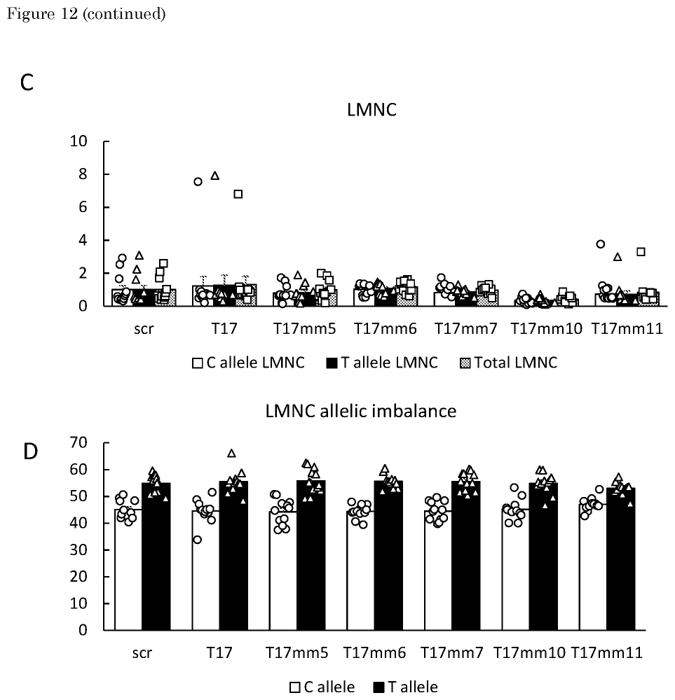

Because both alleles of the SNP could reside on a mutant LMNA allele in different patients, we also targeted the T allele of rs4641 in LMNA, which in our H222P hiPSC-CMs resides on the mutant allele. This revealed one shRNA, shT17, which downregulated the targeted T allele by 53% and the non-targeted allele by 8% (FIG. 11A), while again all other shRNA induced an upregulation of both LMNA alleles. This 53% downregulation of the targeted allele, also resulted in a shift in the allelic balance from 45:55% in the SCR negative control transduced cells to a 61:39% ratio in the shT17 transduced cells, meaning that in these cells the ratio shifted from a small relative overexpression of the mutant allele towards a relative under-expression of the mutant allele by the shT17 (FIG. 11B). Also the shRNAs targeting the T allele of the LMNA splice isoform did not affect allelic imbalance of the LMNC splice isoform. We also added extra mismatches to the shT17 sequence, again at position 5, 6, and 7 in the seed region and position 10, and 11 in the Argonaute recognition region. Interestingly, we observed similar effects as with the addition of these mismatches to the shC17 sequence (FIG. 9). Again the mismatch at position 5 more strongly induced downregulation of the targeted T allele, even up to 83%, but also more strongly induced downregulation of the non-targeted C allele to 44%. The mismatch at position 7 resulted in a downregulation similar to the original T17 sequence in this experiment (20%), but again resulted in a strong upregulation of the non-targeted C allele (FIG. 12A). As a consequence both shRNAs with a mismatch at position 5 and 7 (76:24% and 74:26%) resulted in a stronger effect on the allelic imbalance compared to the original T17 sequence (70:30%; FIG. 12B). Again these shRNAs did not affect the allelic imbalance of LMNC (FIG. 12C/D). Because of the same question whether absolute levels of the mutant allele or the allelic imbalance is the most important factor that determines disease severity in patients and phenotype in our hiPSC-CMs, we also decided to further study shT17, shT17 mm5 and shT17 mm7 and compare their effects on the disease phenotype in hiPSC-CMs.

### Allele-Specific Targeting of Rs4641 in the LMNC Isoform

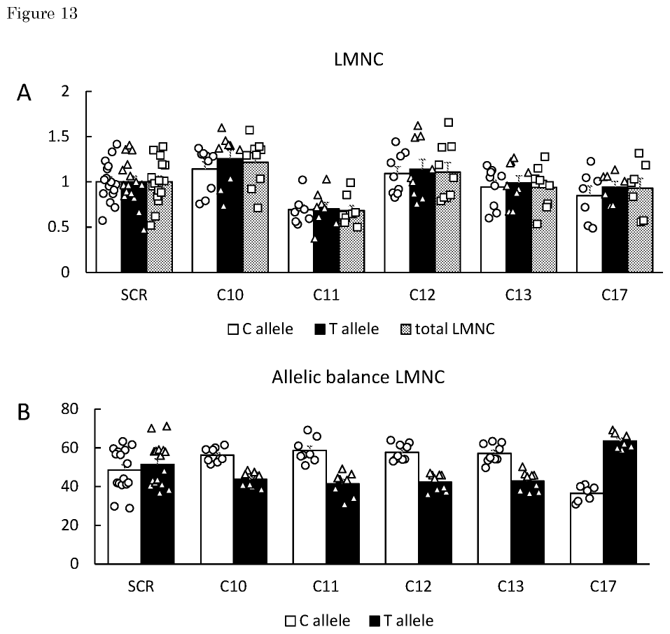

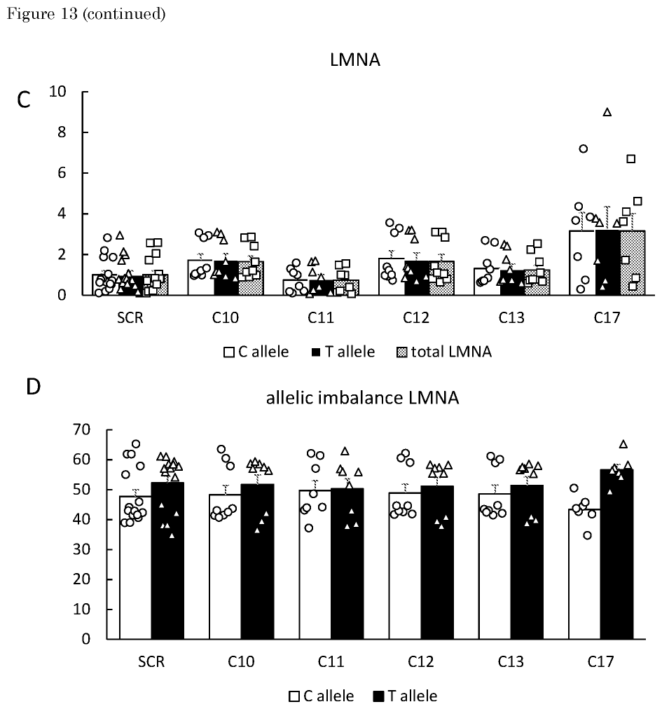

To test the shRNAs targeting LMNC, we lentivirally transduced cardiomyocytes derived from the H222P hiPSC-line with these shRNAs and compared by allele-specific qRT-PCR expression levels of mutant and wildtype LMNC alleles to the expression levels in cells transduced with a scrambled negative control shRNA (Scr). Targeting the C allele of rs4641 in the LMNC splice isoform, which resides in this H222P hiPSC-line on the wildtype LMNC allele, revealed 3 shRNAs, shC11, shC13 and shC17 with a 31%, 6% and 16% knockdown of the C allele respectively (FIG. 13). However, shC11 had also 30% knockdown of the non-targeted T allele and shC17 6%. This resulted in small shifts in allelic balance from 48:51% in the scrambled transduced cells to 59:41% for shC11, 57:43% for shC13 and 36:64% for shC17. We also determined the effects of these shRNAs on the LMNA expression levels and noticed that shC11 also non allele-specifically inhibited the LMNA isoform by 25% and the shC17 increased both the levels of the C and T allele, but the T allele to a larger extend, which therefore also resulted in a slight shift in allelic balance of LMNA (from 48:52% in SCR to 43:57% in shC17). This implies that shC17 might be a good candidate that could affect both splice isoforms. Given the results of the mismatches added to the shC17 targeting the LMNA isoform described above, we speculate that adding the mismatches 5 and 7 to shC17 targeting the LMNC isoform could also further improve allele-specific targeting or allelic imbalance induction of LMNC with a possibility that it immediately also affects LMNA, which might reduce the need to target both splice isoforms separately.

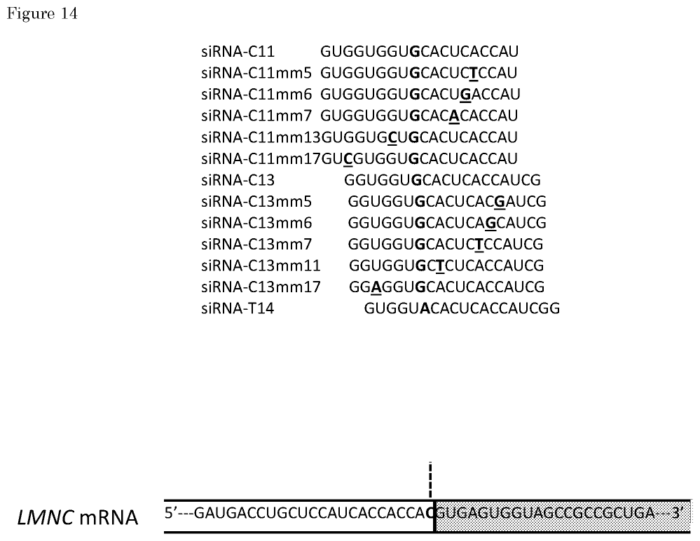

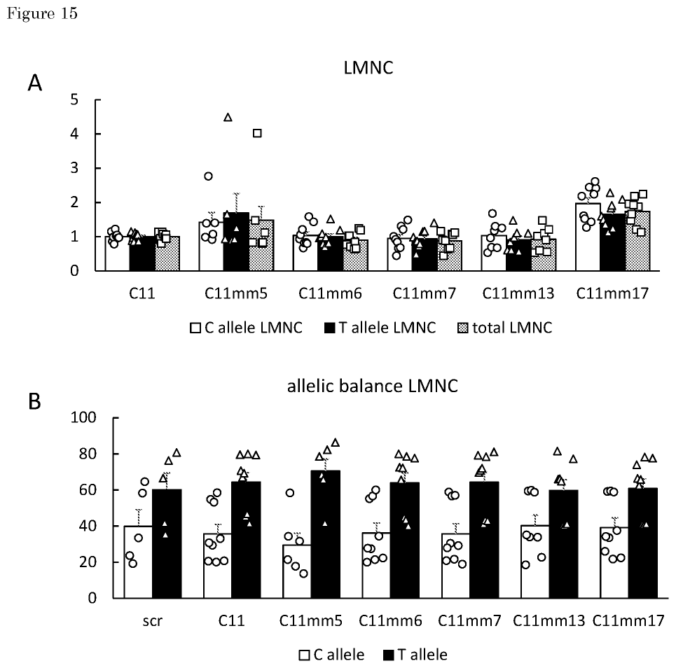

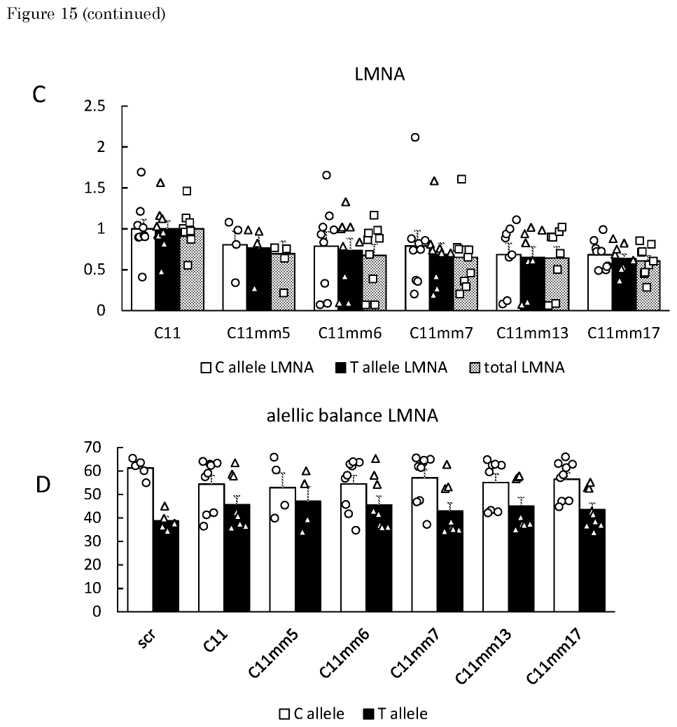

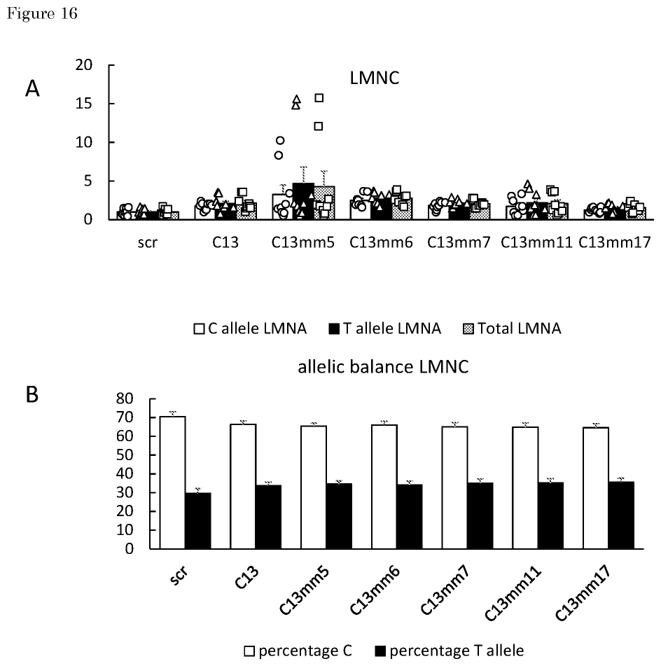

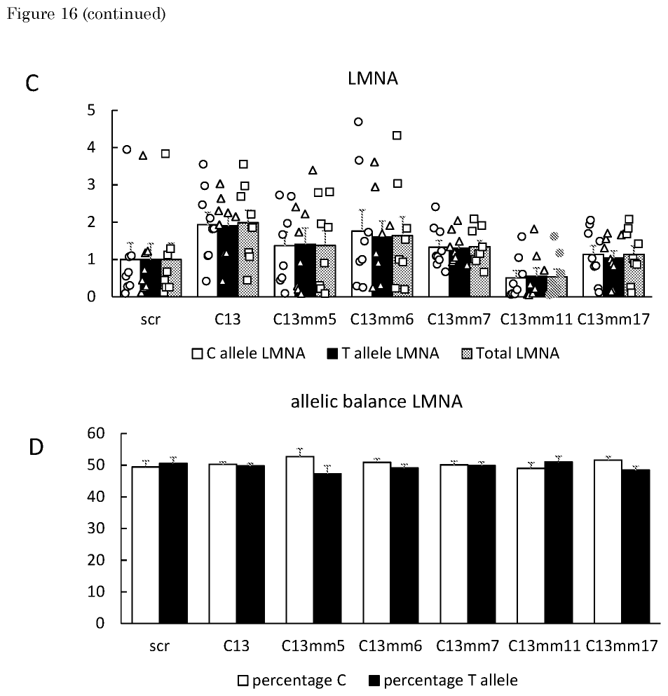

To further improve allele-specific targeting by shC11 and shC13 targeting the C allele of the LMNC isoform we added mismatches to these shRNA sequences (FIG. 14). The mismatches added to shC11, did not result in addition downregulation of the targeted or non-targeted LMNC alleles (FIG. 15), shC11 mm5 resulted in a small upregulation which was stronger in the non-targeted allele, which resulted in a slightly stronger allelic imbalance (40:60% scr; 36:64% shC11; and 30:70% shC11 mm5). The addition of the mismatches to the C13 shRNA sequence did result in a stronger downregulation of the targeted C allele for shC13 mm17 (FIG. 16). However, the downregulation of the non-targeted allele was almost as strong, which resulted in a comparable allelic balance between the original C13 shRNA and the shC13 mm17 (66:34% versus 65:35%). However, depending on the phenotyping results performed on the LMNA targeting shRNAs, if the loss of absolute levels of the mutant allele turns out to be more important than the effect on allelic balance, this shC13 mm 17 might be an interesting candidate for further phenotyping.

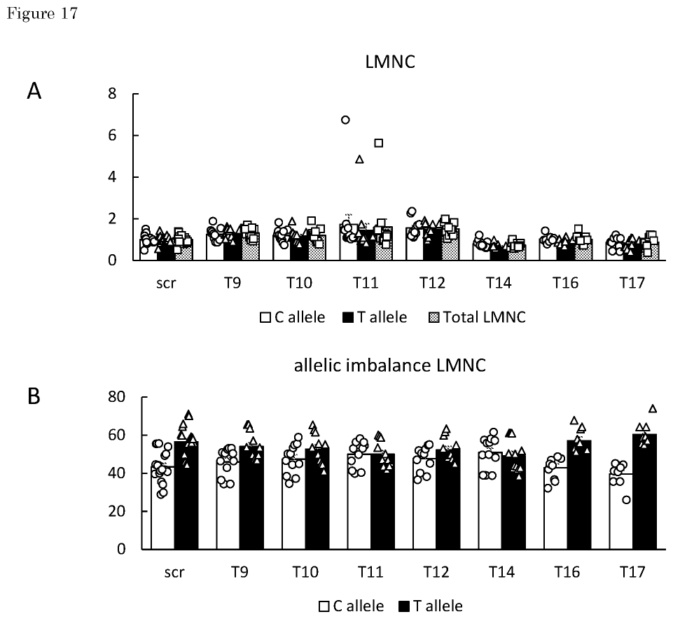

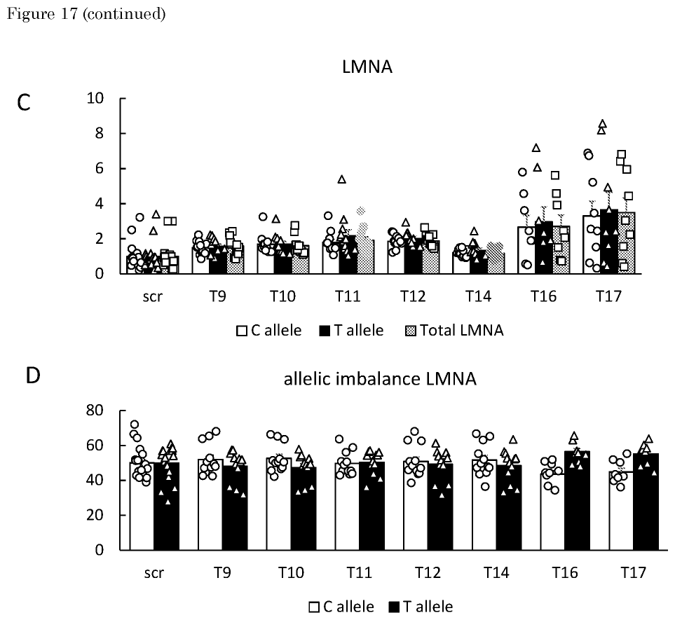

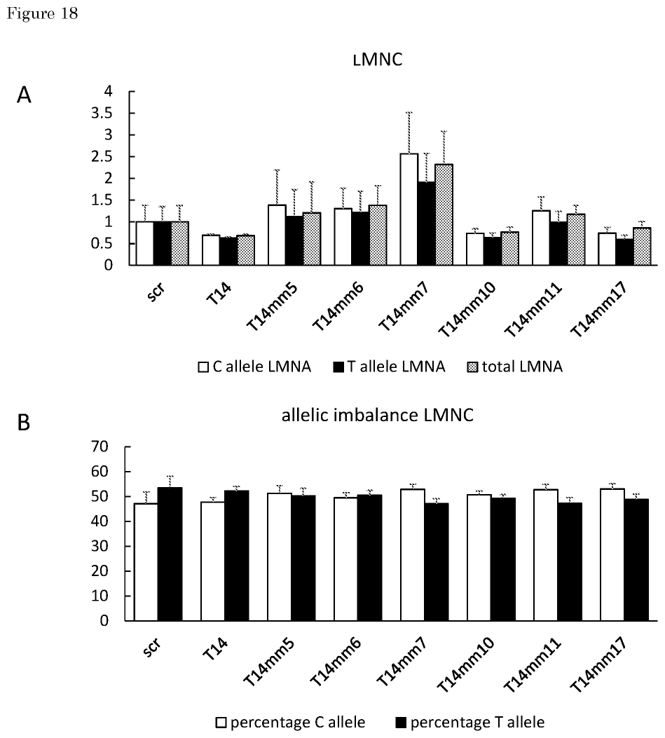

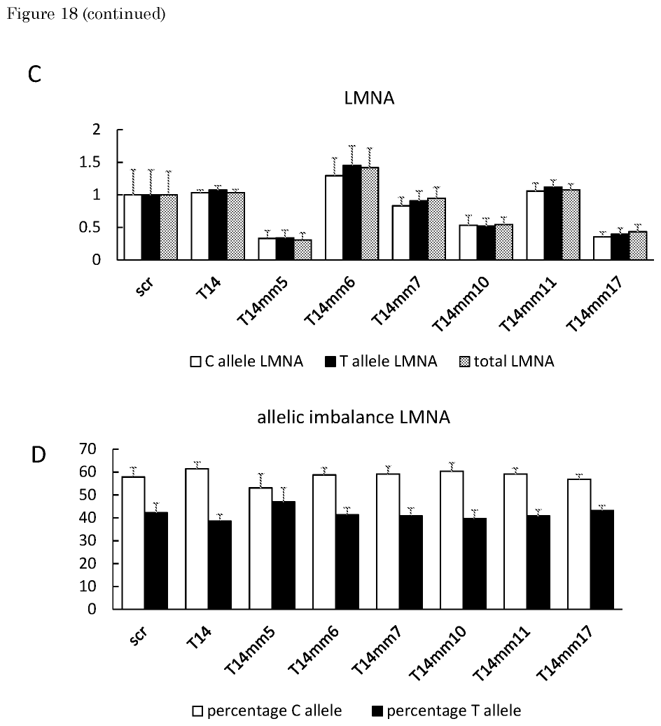

Again because both allele of the rs4641 could reside on the mutant allele in different patients, we also tested the shRNAs targeting the T allele of rs4641 in the LMNC isoform. This revealed two shRNA able to downregulate the targeted T allele by 33% for shT14 and 22% for shT17, while they also downregulated the non-targeted C allele by 20% and 15% respectively. To our surprise, the allelic imbalance for shT17 shifted towards a relative overexpression of the targeted T allele (40:60% vs 43:57% for scr) that was stronger downregulated. The shT14 did shift the allelic imbalance in the expected direction towards a relative under-expression of the targeted T allele (51:49%; FIG. 17). Both shRNAs did not affect the allelic imbalance of the LMNA splice isoform. We did not yet add additional mismatches to the shT17 targeting the LMNC isoform, however, based on the effects observed after addition of mismatches to shT17 targeting the LMNA splice isoform we could imagine that mismatch 5 and 7 could also improve targeting by shT17. We did add additional mismatches to the shT14 (FIG. 18), which revealed no shRNA that was able to more strongly inhibit the targeted T allele or to further affect the allelic imbalance.

TABLE 3
Additional shRNA targeting sequences

| | shRNA | Location | Sense strand | Antisense strand |
|---|---|---|---|---|
| Target | No. | mismatch | (5′-3′) | (5′-3′) |
| C allele | 324 | C17 | CACGTGAGTGGTAGCCGCC | GGCGGCTACCACTCACGTG |
| LMNC | | | SEQ ID NO: 179 | SEQ ID NO: 180 |
| | 327 | C11mm5 | CACCACCACGTGAGaGGTA | TACCtCTCACGTGGTGGTG |
| | | | SEQ ID NO: 181 | SEQ ID NO: 182 |
| | 328 | C11mm6 | CACCACCACGTGAcTGGTA | TACCAgTCACGTGGTGGTG |
| | | | SEQ ID NO: 183 | SEQ ID NO: 184 |
| | 329 | C11mm7 | CACCACCACGTGtGTGGTA | TACCACaCACGTGGTGGTG |
| | | | SEQ ID NO: 185 | SEQ ID NO: 186 |
| | 330 | C11mm13 | CACCACgACGTGAGTGGTA | TACCACTCACGTcGTGGTG |
| | | | SEQ ID NO: 187 | SEQ ID NO: 188 |
| | 331 | C11mm17 | CAgCACCACGTGAGTGGTA | TACCACTCACGTGGTGcTG |
| | | | SEQ ID NO: 189 | SEQ ID NO: 190 |
| | 332 | C13mm5 | CCACCACGTGAGTGcTAGC | GCTAgCACTCACGTGGTGG |
| | | | SEQ ID NO: 191 | SEQ ID NO: 192 |
| | 333 | C13mm6 | CCACCACGTGAGTcGTAGC | GCTACgACTCACGTGGTGG |
| | | | SEQ ID NO: 193 | SEQ ID NO: 194 |
| | 334 | C13mm7 | CCACCACGTGAGaGGTAGC | GCTACCtCTCACGTGGTGG |
| | | | SEQ ID NO: 195 | SEQ ID NO: 196 |
| T allele | 325 | T16 | CCATGTGAGTGGTAGCCGC | GCGGCTACCACTCACATGG |
| LMNC | | | SEQ ID NO: 197 | SEQ ID NO: 198 |
| | 326 | T17 | CATGTGAGTGGTAGCCGCC | GGCGGCTACCACTCACATG |
| | | | SEQ ID NO: 199 | SEQ ID NO: 200 |
| | 342 | T14mm5 | CACCATGTGAGTGGaAGCC | GGCTtCCACTCACATGGTG |
| | | | SEQ ID NO: 201 | SEQ ID NO: 202 |
| | 343 | T14mm6 | CACCATGTGAGTGcTAGCC | GGCTAgCACTCACATGGTG |
| | | | SEQ ID NO: 203 | SEQ ID NO: 204 |
| | 344 | T14mm7 | CACCATGTGAGTcGTAGCC | GGCTACgACTCACATGGTG |
| | | | SEQ ID NO: 205 | SEQ ID NO: 206 |
| | 345 | T14mm10 | CACCATGTGtGTGGTAGCC | GGCTACCACaCACATGGTG |
| | | | SEQ ID NO: 207 | SEQ ID NO: 208 |
| | 346 | T14mm11 | CACCATGTcAGTGGTAGCC | GGCTACCACTgACATGGTG |
| | | | SEQ ID NO: 209 | SEQ ID NO: 210 |
| | | | SEQ ID NO: 200 | |
| | 347 | T14mm17 | CAgCATGTGAGTGGTAGCC | GGCTACCACTCACATGcTG |
| | | | SEQ ID NO: 211 | SEQ ID NO: 212 |
| | 325 | T16 | CCATGTGAGTGGTAGCCGC | GCGGCTACCACTCACATGG |
| | | | SEQ ID NO: 213 | SEQ ID NO: 214 |
| C allele | 314 | C17mm5 | CACGGCTCCCACTGGAGCA | TGCTCCAGTGGGAGCCGTG |
| LMNA | | | SEQ ID NO: 215 | SEQ ID NO: 216 |
| | 315 | C17mm6 | CACGGCTCCCACTCCAGCA | TGCTGGAGTGGGAGCCGTG |
| | | | SEQ ID NO: 217 | SEQ ID NO: 218 |
| | 316 | C17mm7 | CACGGCTCCCACAGCAGCA | TGCTGCTGTGGGAGCCGTG |
| | | | SEQ ID NO: 219 | SEQ ID NO: 220 |
| | 317 | C17mm10 | CACGGCTCCGACTGCAGCA | TGCTGCAGTCGGAGCCGTG |
| | | | SEQ ID NO: 221 | SEQ ID NO: 222 |
| | 318 | C17mm11 | CACGGCTCGCACTGCAGCA | TGCTGCAGTGCGAGCCGTG |
| | | | SEQ ID NO: 223 | SEQ ID NO: 224 |
| T allele | 319 | T17mm5 | CATGGCTCCCACTGGAGCA | TGCTCCAGTGGGAGCCATG |
| LMNA | | | SEQ ID NO: 225 | SEQ ID NO: 226 |
| | 320 | T17mm6 | CATGGCTCCCACTCCAGCA | TGCTGGAGTGGGAGCCATG |
| | | | SEQ ID NO: 227 | SEQ ID NO: 228 |
| | 321 | T17mm7 | CATGGCTCCCACAGCAGCA | TGCTGCTGTGGGAGCCATG |
| | | | SEQ ID NO: 229 | SEQ ID NO: 230 |
| | 322 | T17mm10 | CATGGCTCCGACTGCAGCA | TGCTGCAGTCGGAGCCATG |
| | | | SEQ ID NO: 231 | SEQ ID NO: 232 |
| | 323 | T17mm11 | CATGGCTCGCACTGCAGCA | TGCTGCAGTGCGAGCCATG |
| | | | SEQ ID NO: 233 | SEQ ID NO: 234 |

TABLE 4
Oligo′s used to clone the respective shRNAs in the pLKO-puro plasmid.
The forward primer contains the full shRNA sequence with overhangs
added for cloning. ShRNA starts with the non-capital aa at position 5/6
and ends with the non-capital t-stretch.

| | | | | SEQ | |
|---|---|---|---|---|---|
| | Target | | | ID | |
| Ref | gene | Name | Direction | NO | Sequence |
| 314 | LMNA | C17m5 | fw | 235 | ccggaaCACGGCTCCCACTGGAGCAtcaagacTGCTCCAGTGGGAGCCGTGtttttttg |
| | | | rv | 236 | aattcaaaaaaaCACGGCTCCCACTGGAGCAgtcttgaTGCTCCAGTGGGAGCCGTGtt |
| 315 | LMNA | C17m6 | fw | 237 | ccggaaCACGGCTCCCACTCCAGCAtcaagacTGCTGGAGTGGGAGCCGTGtttttttg |
| | | | rv | 238 | aattcaaaaaaaCACGGCTCCCACTCCAGCAgtcttgaTGCTGGAGTGGGAGCCGTGtt |
| 316 | LMNA | C17m7 | fw | 239 | ccggaaCACGGCTCCCACAGCAGCAtcaagacTGCTGCTGTGGGAGCCGTGtttttttg |
| | | | rv | 240 | aattcaaaaaaaCACGGCTCCCACAGCAGCAgtcttgaTGCTGCTGTGGGAGCCGTGtt |
| 317 | LMNA | C17m10 | fw | 241 | ccggaaCACGGCTCCGACTGCAGCAtcaagacTGCTGCAGTCGGAGCCGTGtttttttg |
| | | | rv | 242 | aattcaaaaaaaCACGGCTCCGACTGCAGCAgtcttgaTGCTGCAGTCGGAGCCGTGtt |
| 318 | LMNA | C17m11 | fw | 243 | ccggaaCACGGCTCGCACTGCAGCAtcaagacTGCTGCAGTGCGAGCCGTGtttttttg |
| | | | rv | 244 | aattcaaaaaaaCACGGCTCGCACTGCAGCAgtcttgaTGCTGCAGTGCGAGCCGTGtt |
| 319 | LMNA | T17m5 | fw | 245 | ccggaaCATGGCTCCCACTGGAGCAtcaagacTGCTCCAGTGGGAGCCATGtttttttg |
| | | | rv | 246 | aattcaaaaaaaCATGGCTCCCACTGGAGCAgtcttgaTGCTCCAGTGGGAGCCATGtt |
| 320 | LMNA | T17m6 | fw | 247 | ccggaaCATGGCTCCCACTCCAGCAtcaagacTGCTGGAGTGGGAGCCATGtttttttg |
| | | | rv | 248 | aattcaaaaaaaCATGGCTCCCACTCCAGCAgtcttgaTGCTGGAGTGGGAGCCATGtt |
| 321 | LMNA | T17m7 | fw | 249 | ccggaaCATGGCTCCCACAGCAGCAtcaagacTGCTGCTGTGGGAGCCATGtttttttg |
| | | | rv | 250 | aattcaaaaaaaCATGGCTCCCACAGCAGCAgtcttgaTGCTGCTGTGGGAGCCATGtt |
| 322 | LMNA | T17m10 | fw | 251 | ccggaaCATGGCTCCGACTGCAGCAtcaagacTGCTGCAGTCGGAGCCATGtttttttg |
| | | | rv | 252 | aattcaaaaaaaCATGGCTCCGACTGCAGCAgtcttgaTGCTGCAGTCGGAGCCATGtt |
| 323 | LMNA | T17m11 | fw | 253 | ccggaaCATGGCTCGCACTGCAGCAtcaagacTGCTGCAGTGCGAGCCATGtttttttg |
| | | | rv | 254 | aattcaaaaaaaCATGGCTCGCACTGCAGCAgtcttgaTGCTGCAGTGCGAGCCATGtt |
| 324 | LMNC | C17 | fw | 255 | ccggaaCACGTGAGTGGTAGCCGCCtcaagacGGCGGCTACCACTCACGTGtttttttg |
| | | | rv | 256 | aattcaaaaaaaCACGTGAGTGGTAGCCGCCgtcttgaGGCGGCTACCACTCACGTGtt |
| 325 | LMNC | T16 | fw | 257 | ccggaaCCATGTGAGTGGTAGCCGCtcaagacGCGGCTACCACTCACATGGtttttttg |
| | | | rv | 258 | aattcaaaaaaaCCATGTGAGTGGTAGCCGCgtcttgaGCGGCTACCACTCACATGGtt |
| 326 | LMNC | T17 | fw | 259 | ccggaaCATGTGAGTGGTAGCCGCCtcaagacGGCGGCTACCACTCACATGtttttttg |
| | | | rv | 260 | aattcaaaaaaaCATGTGAGTGGTAGCCGCCgtcttgaGGCGGCTACCACTCACATGtt |
| 327 | LMNC | C11mm5 | fw | 261 | ccggaaCACCACCACGTGAGaGGTAtcaagacTACCtCTCACGTGGTGGTGtttttttg |
| | | | rv | 262 | aattcaaaaaaaCACCACCACGTGAGaGGTAgtcttgaTACCtCTCACGTGGTGGTGtt |
| 328 | LMNC | C11mm6 | fw | 263 | ccggaaCACCACCACGTGACTGGTAtcaagacTACCAgTCACGTGGTGGTGtttttttg |
| | | | rv | 264 | aattcaaaaaaaCACCACCACGTGACTGGTAgtcttgaTACCAgTCACGTGGTGGTGtt |
| 329 | LMNC | C11mm7 | fw | 265 | ccggaaCACCACCACGTGtGTGGTAtcaagacTACCACaCACGTGGTGGTGtttttttg |
| | | | rv | 266 | aattcaaaaaaaCACCACCACGTGtGTGGTAgtcttgaTACCACaCACGTGGTGGTGtt |
| 330 | LMNC | C11mm13 | fw | 267 | ccggaaCACCACgACGTGAGTGGTAtcaagacTACCACTCACGTcGTGGTGtttttttg |
| | | | rv | 268 | aattcaaaaaaaCACCACgACGTGAGTGGTAgtcttgaTACCACTCACGTcGTGGTGtt |
| 331 | LMNC | C11mm17 | fw | 269 | ccggaaCAgCACCACGTGAGTGGTAtcaagacTACCACTCACGTGGTGcTGtttttttg |
| | | | rv | 270 | aattcaaaaaaaCAgCACCACGTGAGTGGTAgtcttgaTACCACTCACGTGGTGcTGtt |
| 332 | LMNC | C13mm5 | fw | 271 | ccggaaCCACCACGTGAGTGcTAGCtcaagacGCTAgCACTCACGTGGTGGtttttttg |
| | | | rv | 272 | aattcaaaaaaaCCACCACGTGAGTGcTAGCgtcttgaGCTAgCACTCACGTGGTGGtt |
| 333 | LMNC | C13mm6 | fw | 273 | ccggaaCCACCACGTGAGTcGTAGCtcaagacGCTACgACTCACGTGGTGGtttttttg |
| | | | rv | 274 | aattcaaaaaaaCCACCACGTGAGTcGTAGCgtcttgaGCTACgACTCACGTGGTGGtt |
| 334 | LMNC | C13mm7 | fw | 275 | ccggaaCCACCACGTGAGaGGTAGCtcaagacGCTACCtCTCACGTGGTGGtttttttg |
| | | | rv | 276 | aattcaaaaaaaCCACCACGTGAGaGGTAGCgtcttgaGCTACCtCTCACGTGGTGGtt |
| 335 | LMNC | C13mm11 | fw | 277 | ccggaaCCACCACGaGAGTGGTAGCtcaagacGCTACCACTCtCGTGGTGGtttttttg |
| | | | rv | 278 | aattcaaaaaaaCCACCACGaGAGTGGTAGCgtcttgaGCTACCACTCtCGTGGTGGtt |
| 336 | LMNC | C13mm17 | fw | 279 | ccggaaCCtCCACGTGAGTGGTAGCtcaagacGCTACCACTCACGTGGaGGtttttttg |
| | | | rv | 280 | aattcaaaaaaaCCtCCACGTGAGTGGTAGCgtcttgaGCTACCACTCACGTGGaGGtt |
| 342 | LMNC | T14mm5 | fw | 281 | ccggaaCACCATGTGAGTGGaAGCCtcaagacGGCTtCCACTCACATGGTGtttttttg |
| | | | rv | 282 | aattcaaaaaaaCACCATGTGAGTGGaAGCCgtcttgaGGCTtCCACTCACATGGTGtt |
| 343 | LMNC | T14mm6 | fw | 283 | ccggaaCACCATGTGAGTGcTAGCCtcaagacGGCTAgCACTCACATGGTGtttttttg |
| | | | rv | 284 | aattcaaaaaaaCACCATGTGAGTGcTAGCCgtcttgaGGCTAgCACTCACATGGTGtt |
| 344 | LMNC | T14mm7 | fw | 285 | ccggaaCACCATGTGAGTcGTAGCCtcaagacGGCTACgACTCACATGGTGtttttttg |
| | | | rv | 286 | aattcaaaaaaaCACCATGTGAGTcGTAGCCgtcttgaGGCTACgACTCACATGGTGtt |
| 345 | LMNC | T14mm10 | fw | 287 | ccggaaCACCATGTGtGTGGTAGCCtcaagacGGCTACCACaCACATGGTGtttttttg |
| | | | rv | 288 | aattcaaaaaaaCACCATGTGtGTGGTAGCCgtcttgaGGCTACCACaCACATGGTGtt |
| 346 | LMNC | T14mm11 | fw | 289 | ccggaaCACCATGTcAGTGGTAGCCtcaagacGGCTACCACTgACATGGTGtttttttg |
| | | | rv | 290 | aattcaaaaaaaCACCATGTcAGTGGTAGCCgtcttgaGGCTACCACTCAgATGGTGtt |
| 347 | LMNC | T14mm17 | fw | 291 | ccggaaCAgCATGTGAGTGGTAGCCtcaagacGGCTACCACTCACATGcTGtttttttg |
| | | | rv | 292 | aattcaaaaaaaCAgCATGTGAGTGGTAGCCgtcttgaGGCTACCACTCACATGcTGtt |

Ref: reference number.
Fw: forward.
Rv: reversed.

1. An isolated antisense molecule capable of inhibiting or reducing allele-specific expression of an LMNA gene in a mammalian cell, wherein said antisense molecule comprises an antisense nucleic acid strand which is substantially complementary to a target region of a transcript encoded by the LMNA gene, wherein said antisense nucleic acid strand is at least complementary to SNP rs538089, rs505058 or rs4641 in said target region.

2. The isolated antisense molecule of claim 1 selected from:
a double stranded nucleic acid (dsNA) or a chemically modified version thereof, or
an antisense oligonucleotide (ASO).

3. The isolated antisense molecule according to claim 1, wherein said target region comprises the nucleic acid sequence
(SEQ ID NO: 1)
GAUGACCUGCUCCAUCACCACCACGUGAGUGGUAGCCGCCGCUGA,
(SEQ ID NO: 2)
GAUGACCUGCUCCAUCACCACCAUGUGAGUGGUAGCCGCCGCUGA,
(SEQ ID NO: 3)
GAUGACCUGCUCCAUCACCACCACGGCUCCCACUGCAGCAGCUC,
or
(SEQ ID NO: 4)
GAUGACCUGCUCCAUCACCACCAUGGCUCCCACUGCAGCAGCUC
or a sequence being at least 80% homologous thereto.

4. The isolated antisense molecule according to claim 1, wherein said antisense nucleic acid strand comprises a nucleic acid sequence having a consecutive strand of at least 12 nucleotides selected from SEQ ID NO:151, SEQ ID NO: 152, SEQ ID NO: 153 or SEQ ID NO:154.

5. The isolated antisense molecule of claim 1, wherein said antisense nucleic acid comprises the nucleic acid sequence selected from SEQ ID NO:71, SEQ ID NO:72, SEQ ID NO: 73 or SEQ ID NO:74.

6. The isolated antisense molecule of claim 1, comprising the antisense nucleic acid of SEQ ID NO: 73 or SEQ ID NO:74.

7. The isolated antisense molecule according to claim 1, wherein said antisense molecule comprises a sense nucleic acid strand of 15-30 nucleotides in length; and wherein the sense nucleic acid strand is complementary to said antisense region and wherein the sense and antisense nucleic acid strands form a duplex region.

8. The isolated antisense molecule according to claim 1, wherein the antisense strand and the sense strand are operably linked by means of an RNA loop strand to form a hairpin structure comprising a duplex structure and a loop structure.

9. A nucleic acid encoding the isolated antisense molecule of claim 1.

10. An expression cassette comprising a nucleic acid encoding said antisense molecule of claim 1.

11. An expression vector comprising the expression cassette of claim 10.

12. A pharmaceutical composition comprising the isolated antisense molecule of claim 1 and a pharmaceutically acceptable carrier.

13. A method of treating an individual in need thereof comprising administering to the individual the isolated antisense molecule according to claim 1.

14. A method of treating an individual for heart failure comprising administering to the individual the isolated antisense molecule according to claim 1.

15. A method for selecting an antisense molecule which is suitable for the medical treatment of a disease caused by an autosomal dominant mutation present in a mutant allele of a gene, said method comprising:
providing a nucleic acid sample of a subject suffering from a disease which is caused by an autosomal dominant mutation present in a mutant allele of a gene,
determining the heterozygosity of the mutant allele of said gene which is responsible for said disease,
determining the presence of a heterozygous SNP in said gene,
determining the nucleic acid sequence of a target region comprising the heterozygous SNP in a transcript of the mutant allele of said gene,
selecting an isolated antisense molecule which is complementary to said target region.

16. An in vitro method for inhibiting or reducing the expression of a mutant LMNA allele in a cell, comprising the following steps:
introducing into the cell an antisense molecule of claim 1 which, upon introduction into a cell expressing a LMNA mutant allele, inhibits or reduces expression of the LMNA mutant allele; and
maintaining the cell produced in step (a) for a time sufficient to obtain degradation of the mRNA transcript of the LMNA mutant allele, thereby inhibiting or reducing expression of the LMNA mutant allele in the cell.

17. A pharmaceutical composition comprising the expression cassette of claim 10 and a pharmaceutically acceptable carrier.

18. A method of treating an individual in need thereof comprising administering to the individual the expression cassette of claim 10.

19. A pharmaceutical composition comprising the expression vector of claim 11 and a pharmaceutically acceptable carrier.

20. A method of treating an individual in need thereof comprising administering to the individual the expression vector of claim 11.

21. A method of treating an individual in need thereof comprising administering to the individual the pharmaceutical composition according to claim 12.


SEQUENCE LISTING (292 sequences)

SEQ ID NO: 1 - RNA, 45 nt, synthetic construct
  misc_feature 1..45: note = target nucleic acid 1
  source 1..45: mol_type = other RNA
gatgacctgctccatcaccaccacgtgagtggtagccgccgctga

SEQ ID NO: 2 - RNA, 45 nt, synthetic construct
  misc_feature 1..45: note = target sequence 2
  source 1..45: mol_type = other RNA
gatgacctgctccatcaccaccatgtgagtggtagccgccgctga

SEQ ID NO: 3 - RNA, 44 nt, synthetic construct
  misc_feature 1..44: note = target sequence 3
  source 1..44: mol_type = other RNA
gatgacctgctccatcaccaccacggctcccactgcagcagctc

SEQ ID NO: 4 - RNA, 44 nt, synthetic construct
  misc_feature 1..44: note = target sequence 4
  source 1..44: mol_type = other RNA
gatgacctgctccatcaccaccatggctcccactgcagcagctc

SEQ ID NO: 5 - RNA, 37 nt, synthetic construct
  misc_feature 1..37: note = target sequence 5
  source 1..37: mol_type = other RNA
cctgctccatcaccaccacgtgagtggtagccgccgc

SEQ ID NO: 6 - RNA, 37 nt, synthetic construct
  misc_feature 1..37: note = target sequence 6
  source 1..37: mol_type = other RNA
cctgctccatcaccaccatgtgagtggtagccgccgc

SEQ ID NO: 7 - RNA, 37 nt, synthetic construct
  misc_feature 1..37: note = target sequence 7
  source 1..37: mol_type = other RNA
cctgctccatcaccaccacggctcccactgcagcagc

SEQ ID NO: 8 - RNA, 37 nt, synthetic construct
  misc_feature 1..37: note = target sequence 8
  source 1..37: mol_type = other RNA
cctgctccatcaccaccatggctcccactgcagcagc

SEQ ID NO: 9 - DNA, 19 bp, synthetic construct
  misc_feature 1..19: note = 175 sense
  source 1..19: mol_type = other DNA
gaaatgtactgcgtggaga

SEQ ID NO: 10 - DNA, 19 bp, synthetic construct
  misc_feature 1..19: note = 175 antisense
  source 1..19: mol_type = other DNA
tctccacgcagtacatttc

SEQ ID NO: 11 - DNA, 19 bp, synthetic construct
  misc_feature 1..19: note = 209 sense
  source 1..19: mol_type = other DNA
tcaccaccacgtgagtggt

SEQ ID NO: 12 - DNA, 19 bp, synthetic construct
  misc_feature 1..19: note = 209 antisense
  source 1..19: mol_type = other DNA
accactcacgtggtggtga

SEQ ID NO: 13 - DNA, 19 bp, synthetic construct
  misc_feature 1..19: note = 210 sense
  source 1..19: mol_type = other DNA
caccaccacgtgagtggta

SEQ ID NO: 14 - DNA, 19 bp, synthetic construct
  misc_feature 1..19: note = 210 antisense
  source 1..19: mol_type = other DNA
taccactcacgtggtggtg

SEQ ID NO: 15 - DNA, 19 bp, synthetic construct
  misc_feature 1..19: note = 211 sense
  source 1..19: mol_type = other DNA
accaccacgtgagtggtag

SEQ ID NO: 16 - DNA, 19 bp, synthetic construct
  misc_feature 1..19: note = 211 antisense
  source 1..19: mol_type = other DNA
ctaccactcacgtggtggt

SEQ ID NO: 17 - DNA, 19 bp, synthetic construct
  misc_feature 1..19: note = 212 sense
  source 1..19: mol_type = other DNA
ccaccacgtgagtggtagc

SEQ ID NO: 18 - DNA, 19 bp, synthetic construct
  misc_feature 1..19: note = 212 antisense
  source 1..19: mol_type = other DNA
gctaccactcacgtggtgg

SEQ ID NO: 19 - DNA, 19 bp, synthetic construct
  misc_feature 1..19: note = 213 sense
  source 1..19: mol_type = other DNA
atcaccaccatgtgagtgg

SEQ ID NO: 20 - DNA, 19 bp, synthetic construct
  misc_feature 1..19: note = 213 antisense
  source 1..19: mol_type = other DNA
ccactcacatggtggtgat

SEQ ID NO: 21 - DNA, 19 bp, synthetic construct
  misc_feature 1..19: note = 214 sense
  source 1..19: mol_type = other DNA
tcaccaccatgtgagtggt

SEQ ID NO: 22 - DNA, 19 bp, synthetic construct
  misc_feature 1..19: note = 214 antisense
  source 1..19: mol_type = other DNA
accactcacatggtggtga

SEQ ID NO: 23 - DNA, 19 bp, synthetic construct
  misc_feature 1..19: note = 215 sense
  source 1..19: mol_type = other DNA
caccaccatgtgagtggta

SEQ ID NO: 24 - DNA, 19 bp, synthetic construct
  misc_feature 1..19: note = 215 antisense
  source 1..19: mol_type = other DNA
taccactcacatggtggtg

SEQ ID NO: 25 - DNA, 19 bp, synthetic construct
  misc_feature 1..19: note = 216 sense
  source 1..19: mol_type = other DNA
accaccatgtgagtggtag

SEQ ID NO: 26 - DNA, 19 bp, synthetic construct
  misc_feature 1..19: note = 216 antisense
  source 1..19: mol_type = other DNA
ctaccactcacatggtggt

SEQ ID NO: 27 - DNA, 19 bp, synthetic construct
  misc_feature 1..19: note = 217 sense
  source 1..19: mol_type = other DNA
caccatgtgagtggtagcc

SEQ ID NO: 28 - DNA, 19 bp, synthetic construct
  misc_feature 1..19: note = 217 antisense
  source 1..19: mol_type = other DNA
ggctaccactcacatggtg

SEQ ID NO: 29 - DNA, 19 bp, synthetic construct
  misc_feature 1..19: note = 230 sense
  source 1..19: mol_type = other DNA
tcaccaccacggctcccac

SEQ ID NO: 30 - DNA, 19 bp, synthetic construct
  misc_feature 1..19: note = 230 antisense
  source 1..19: mol_type = other DNA
gtgggagccgtggtggtga

SEQ ID NO: 31 - DNA, 19 bp, synthetic construct
  misc_feature 1..19: note = 231 sense
  source 1..19: mol_type = other DNA
caccaccacggctcccact

SEQ ID NO: 32 - DNA, 19 bp, synthetic construct
  misc_feature 1..19: note = 231 antisense
  source 1..19: mol_type = other DNA
agtgggagccgtggtggtg

SEQ ID NO: 33 - DNA, 19 bp, synthetic construct
  misc_feature 1..19: note = 232 sense
  source 1..19: mol_type = other DNA
accaccacggctcccactg

SEQ ID NO: 34 - DNA, 19 bp, synthetic construct
  misc_feature 1..19: note = 232 antisense
  source 1..19: mol_type = other DNA
cagtgggagccgtggtggt

SEQ ID NO: 35 - DNA, 19 bp, synthetic construct
  misc_feature 1..19: note = 233 sense
  source 1..19: mol_type = other DNA
ccaccacggctcccactgc

SEQ ID NO: 36 - DNA, 19 bp, synthetic construct
  misc_feature 1..19: note = 233 antisense
  source 1..19: mol_type = other DNA
gcagtgggagccgtggtgg

SEQ ID NO: 37 - DNA, 19 bp, synthetic construct
  misc_feature 1..19: note = 234 sense
  source 1..19: mol_type = other DNA
caccacggctcccactgca

SEQ ID NO: 38 - DNA, 19 bp, synthetic construct
  misc_feature 1..19: note = 234 antisense
  source 1..19: mol_type = other DNA
tgcagtgggagccgtggtg

SEQ ID NO: 39 - DNA, 19 bp, synthetic construct
  misc_feature 1..19: note = 235 sense
  source 1..19: mol_type = other DNA
cacggctcccactgcagca

SEQ ID NO: 40 - DNA, 19 bp, synthetic construct
  misc_feature 1..19: note = 235 antisense
  source 1..19: mol_type = other DNA
tgctgcagtgggagccgtg

SEQ ID NO: 41 - DNA, 19 bp, synthetic construct
  misc_feature 1..19: note = 236 sense
  source 1..19: mol_type = other DNA
atcaccaccatggctccca

SEQ ID NO: 42 - DNA, 19 bp, synthetic construct
  misc_feature 1..19: note = 236 antisense
  source 1..19: mol_type = other DNA
tgggagccatggtggtgat

SEQ ID NO: 43 - DNA, 19 bp, synthetic construct
  misc_feature 1..19: note = 237 sense
  source 1..19: mol_type = other DNA
tcaccaccatggctcccac

SEQ ID NO: 44 - DNA, 19 bp, synthetic construct
  misc_feature 1..19: note = 237 antisense
  source 1..19: mol_type = other DNA
gtgggagccatggtggtga

SEQ ID NO: 45 - DNA, 19 bp, synthetic construct
  misc_feature 1..19: note = 238 sense
  source 1..19: mol_type = other DNA
caccaccatggctcccact

SEQ ID NO: 46 - DNA, 19 bp, synthetic construct
  misc_feature 1..19: note = 238 antisense
  source 1..19: mol_type = other DNA
agtgggagccatggtggtg

SEQ ID NO: 47 - DNA, 19 bp, synthetic construct
  misc_feature 1..19: note = 239 sense
  source 1..19: mol_type = other DNA
accaccatggctcccactg

SEQ ID NO: 48 - DNA, 19 bp, synthetic construct
  misc_feature 1..19: note = 239 antisense
  source 1..19: mol_type = other DNA
cagtgggagccatggtggt

SEQ ID NO: 49 - DNA, 19 bp, synthetic construct
  misc_feature 1..19: note = 240 sense
  source 1..19: mol_type = other DNA
caccatggctcccactgca

SEQ ID NO: 50 - DNA, 19 bp, synthetic construct
  misc_feature 1..19: note = 240 antisense
  source 1..19: mol_type = other DNA
tgcagtgggagccatggtg

SEQ ID NO: 51 - DNA, 19 bp, synthetic construct
  misc_feature 1..19: note = 241 sense
  source 1..19: mol_type = other DNA
catggctcccactgcagca

SEQ ID NO: 52 - DNA, 19 bp, synthetic construct
  misc_feature 1..19: note = 241 antisense
  source 1..19: mol_type = other DNA
tgctgcagtgggagccatg

SEQ ID NO: 53 - DNA, 20 bp, synthetic construct
  misc_feature 1..20: note = C LMNA FW
  source 1..20: mol_type = other DNA
acctgctccatcaccaccac

SEQ ID NO: 54 - DNA, 20 bp, synthetic construct
  misc_feature 1..20: note = C LMNA REV
  source 1..20: mol_type = other DNA
agccgctggcagatgccttg

SEQ ID NO: 55 - DNA, 20 bp, synthetic construct
  misc_feature 1..20: note = T LMNA FW
  source 1..20: mol_type = other DNA
acctgctccatcaccaccat

SEQ ID NO: 56 - DNA, 20 bp, synthetic construct
  misc_feature 1..20: note = T LMNA REV
  source 1..20: mol_type = other DNA
agccgctggcagatgccttg

SEQ ID NO: 57 - DNA, 18 bp, synthetic construct
  misc_feature 1..18: note = Total LMNA FW
  source 1..18: mol_type = other DNA
acctgctccatcaccacc

SEQ ID NO: 58 - DNA, 20 bp, synthetic construct
  misc_feature 1..20: note = Total LMNA REV
  source 1..20: mol_type = other DNA
agccgctggcagatgccttg

SEQ ID NO: 59 - DNA, 20 bp, synthetic construct
  misc_feature 1..20: note = C LMNC FW
  source 1..20: mol_type = other DNA
acctgctccatcaccaccac

SEQ ID NO: 60 - DNA, 18 bp, synthetic construct
  misc_feature 1..18: note = C LMNC REV
  source 1..18: mol_type = other DNA
tggccccagtgcaggctc

SEQ ID NO: 61 - DNA, 20 bp, synthetic construct
  misc_feature 1..20: note = T LMNC FW
  source 1..20: mol_type = other DNA
acctgctccatcaccaccat

SEQ ID NO: 62 - DNA, 18 bp, synthetic construct
  misc_feature 1..18: note = T LMNC REV
  source 1..18: mol_type = other DNA
tggccccagtgcaggctc

SEQ ID NO: 63 - DNA, 18 bp, synthetic construct
  misc_feature 1..18: note = Total LMNC FW
  source 1..18: mol_type = other DNA
acctgctccatcaccacc

SEQ ID NO: 64 - DNA, 18 bp, synthetic construct
  misc_feature 1..18: note = Total LMNC REV
  source 1..18: mol_type = other DNA
tggccccagtgcaggctc

SEQ ID NO: 65 - DNA, 21 bp, synthetic construct
  misc_feature 1..21: note = GAPDH FW
  source 1..21: mol_type = other DNA
acccactcctccacctttgac

SEQ ID NO: 66 - DNA, 22 bp, synthetic construct
  misc_feature 1..22: note = GAPDH REV
  source 1..22: mol_type = other DNA
accctgttgctgtagccaaatt

SEQ ID NO: 67 - DNA, 21 bp, synthetic construct
  misc_feature 1..21: note = HPRT FW
  source 1..21: mol_type = other DNA
tgacactggcaaaacaatgca

SEQ ID NO: 68 - DNA, 21 bp, synthetic construct
  misc_feature 1..21: note = HPRT REV
  source 1..21: mol_type = other DNA
ggtccttttcaccagcaagct

SEQ ID NO: 69 - DNA, 21 bp, synthetic construct
  misc_feature 1..21: note = TBP FW
  source 1..21: mol_type = other DNA
gctcacccaccaacaatttag

SEQ ID NO: 70 - DNA, 22 bp, synthetic construct
  misc_feature 1..22: note = TBP REV
  source 1..22: mol_type = other DNA
tctgctctgactttagcacctg

SEQ ID NO: 71 - RNA, 19 nt, synthetic construct
  misc_feature 1..19: note = C11-lamin C
  source 1..19: mol_type = other RNA
taccactcacgtggtggtg

SEQ ID NO: 72 - RNA, 19 nt, synthetic construct
  misc_feature 1..19: note = T14-lamin C
  source 1..19: mol_type = other RNA
ggctaccactcacatggtg

SEQ ID NO: 73 - RNA, 19 nt, synthetic construct
  misc_feature 1..19: note = C17-lamin A
  source 1..19: mol_type = other RNA
tgctgcagtgggagccgtg

SEQ ID NO: 74 - RNA, 19 nt, synthetic construct
  misc_feature 1..19: note = T17-lamin A
  source 1..19: mol_type = other RNA
tgctgcagtgggagccatg

SEQ ID NO: 75 - DNA, 59 bp, synthetic construct
  misc_feature 1..59: note = LMNC-C1
  source 1..59: mol_type = other DNA
ccggaacctgctccatcaccaccactcaagacgtggtggtgatggagcaggtttttttg

SEQ ID NO: 76 - DNA, 59 bp, synthetic construct
  misc_feature 1..59: note = LMNC-C2
  source 1..59: mol_type = other DNA
ccggaactgctccatcaccaccacgtcaagaccgtggtggtgatggagcagtttttttg

SEQ ID NO: 77 - DNA, 59 bp, synthetic construct
  misc_feature 1..59: note = LMNC-C3
  source 1..59: mol_type = other DNA
ccggaatgctccatcaccaccacgttcaagacacgtggtggtgatggagcatttttttg

SEQ ID NO: 78 - DNA, 59 bp, synthetic construct
  misc_feature 1..59: note = LMNC-C4
  source 1..59: mol_type = other DNA
ccggaagctccatcaccaccacgtgtcaagaccacgtggtggtgatggagctttttttg

SEQ ID NO: 79 - DNA, 59 bp, synthetic construct
  misc_feature 1..59: note = LMNC-C5
  source 1..59: mol_type = other DNA
ccggaactccatcaccaccacgtgatcaagactcacgtggtggtgatggagtttttttg

SEQ ID NO: 80 - DNA, 59 bp, synthetic construct
  misc_feature 1..59: note = LMNC-C6
  source 1..59: mol_type = other DNA
ccggaatccatcaccaccacgtgagtcaagacctcacgtggtggtgatggatttttttg

SEQ ID NO: 81 - DNA, 59 bp, synthetic construct
  misc_feature 1..59: note = LMNC-C7
  source 1..59: mol_type = other DNA
ccggaaccatcaccaccacgtgagttcaagacactcacgtggtggtgatggtttttttg

SEQ ID NO: 82 - DNA, 59 bp, synthetic construct
  misc_feature 1..59: note = LMNC-C8
  source 1..59: mol_type = other DNA
ccggaacatcaccaccacgtgagtgtcaagaccactcacgtggtggtgatgtttttttg

SEQ ID NO: 83 - DNA, 59 bp, synthetic construct
  misc_feature 1..59: note = LMNC-C9
  source 1..59: mol_type = other DNA
ccggaaatcaccaccacgtgagtggtcaagacccactcacgtggtggtgattttttttg

SEQ ID NO: 84 - DNA, 59 bp, synthetic construct
  misc_feature 1..59: note = LMNC-C10
  source 1..59: mol_type = other DNA
ccggaatcaccaccacgtgagtggttcaagacaccactcacgtggtggtgatttttttg

SEQ ID NO: 85 - DNA, 59 bp, synthetic construct
  misc_feature 1..59: note = LMNC-C11
  source 1..59: mol_type = other DNA
ccggaacaccaccacgtgagtggtatcaagactaccactcacgtggtggtgtttttttg

SEQ ID NO: 86 - DNA, 59 bp, synthetic construct
  misc_feature 1..59: note = LMNC-C12
  source 1..59: mol_type = other DNA
ccggaaaccaccacgtgagtggtagtcaagacctaccactcacgtggtggttttttttg

SEQ ID NO: 87 - DNA, 59 bp, synthetic construct
  misc_feature 1..59: note = LMNC-C13
  source 1..59: mol_type = other DNA
ccggaaccaccacgtgagtggtagctcaagacgctaccactcacgtggtggtttttttg

SEQ ID NO: 88 - DNA, 59 bp, synthetic construct
  misc_feature 1..59: note = LMNC-C14
  source 1..59: mol_type = other DNA
ccggaacaccacgtgagtggtagcctcaagacggctaccactcacgtggtgtttttttg

SEQ ID NO: 89 - DNA, 59 bp, synthetic construct
  misc_feature 1..59: note = LMNC-C15
  source 1..59: mol_type = other DNA
ccggaaaccacgtgagtggtagccgtcaagaccggctaccactcacgtggttttttttg

SEQ ID NO: 90 - DNA, 59 bp, synthetic construct
  misc_feature 1..59: note = LMNC-C16
  source 1..59: mol_type = other DNA
ccggaaccacgtgagtggtagccgctcaagacgcggctaccactcacgtggtttttttg

SEQ ID NO: 91 - DNA, 59 bp, synthetic construct
  misc_feature 1..59: note = LMNC-C17
  source 1..59: mol_type = other DNA
ccggaacacgtgagtggtagccgcctcaagacggcggctaccactcacgtgtttttttg

SEQ ID NO: 92 - DNA, 59 bp, synthetic construct
  misc_feature 1..59: note = LMNC-C18
  source 1..59: mol_type = other DNA
ccggaaacgtgagtggtagccgccgtcaagaccggcggctaccactcacgttttttttg

SEQ ID NO: 93 - DNA, 59 bp, synthetic construct
  misc_feature 1..59: note = LMNC-C19
  source 1..59: mol_type = other DNA
ccggaacgtgagtggtagccgccgctcaagacgcggcggctaccactcacgtttttttg

SEQ ID NO: 94 - DNA, 59 bp, synthetic construct
  misc_feature 1..59: note = LMNC-T1
  source 1..59: mol_type = other DNA
ccggaacctgctccatcaccaccattcaagacatggtggtgatggagcaggtttttttg

SEQ ID NO: 95 - DNA, 59 bp, synthetic construct
  misc_feature 1..59: note = LMNC-T2
  source 1..59: mol_type = other DNA
ccggaactgctccatcaccaccatgtcaagaccatggtggtgatggagcagtttttttg

SEQ ID NO: 96 - DNA, 59 bp, synthetic construct
  misc_feature 1..59: note = LMNC-T3
  source 1..59: mol_type = other DNA
ccggaatgctccatcaccaccatgttcaagacacatggtggtgatggagcatttttttg

SEQ ID NO: 97 - DNA, 59 bp, synthetic construct
  misc_feature 1..59: note = LMNC-T4
  source 1..59: mol_type = other DNA
ccggaagctccatcaccaccatgtgtcaagaccacatggtggtgatggagctttttttg

SEQ ID NO: 98 - DNA, 59 bp, synthetic construct
  misc_feature 1..59: note = LMNC-T5
  source 1..59: mol_type = other DNA
ccggaactccatcaccaccatgtgatcaagactcacatggtggtgatggagtttttttg

SEQ ID NO: 99 - DNA, 59 bp, synthetic construct
  misc_feature 1..59: note = LMNC-T6
  source 1..59: mol_type = other DNA
ccggaatccatcaccaccatgtgagtcaagacctcacatggtggtgatggatttttttg

SEQ ID NO: 100 - DNA, 59 bp, synthetic construct
  misc_feature 1..59: note = LMNC-T7
  source 1..59: mol_type = other DNA
ccggaaccatcaccaccatgtgagttcaagacactcacatggtggtgatggtttttttg

SEQ ID NO: 101 - DNA, 59 bp, synthetic construct
  misc_feature 1..59: note = LMNC-T8
  source 1..59: mol_type = other DNA
ccggaacatcaccaccatgtgagtgtcaagaccactcacatggtggtgatgtttttttg

SEQ ID NO: 102 - DNA, 59 bp, synthetic construct
  misc_feature 1..59: note = LMNC-T9
  source 1..59: mol_type = other DNA
ccggaaatcaccaccatgtgagtggtcaagacccactcacatggtggtgattttttttg

SEQ ID NO: 103 - DNA, 59 bp, synthetic construct
  misc_feature 1..59: note = LMNC-T10
  source 1..59: mol_type = other DNA
ccggaatcaccaccatgtgagtggttcaagacaccactcacatggtggtgatttttttg

SEQ ID NO: 104 - DNA, 59 bp, synthetic construct
  misc_feature 1..59: note = LMNC-T11
  source 1..59: mol_type = other DNA
ccggaacaccaccatgtgagtggtatcaagactaccactcacatggtggtgtttttttg

SEQ ID NO: 105 - DNA, 59 bp, synthetic construct
  misc_feature 1..59: note = LMNC-T12
  source 1..59: mol_type = other DNA
ccggaaaccaccatgtgagtggtagtcaagacctaccactcacatggtggttttttttg

SEQ ID NO: 106 - DNA, 59 bp, synthetic construct
  misc_feature 1..59: note = LMNC-T13
  source 1..59: mol_type = other DNA
ccggaaccaccatgtgagtggtagctcaagacgctaccactcacatggtggtttttttg

SEQ ID NO: 107 - DNA, 59 bp, synthetic construct
  misc_feature 1..59: note = LMNC-T14
  source 1..59: mol_type = other DNA
ccggaacaccatgtgagtggtagcctcaagacggctaccactcacatggtgtttttttg

SEQ ID NO: 108 - DNA, 59 bp, synthetic construct
  misc_feature 1..59: note = LMNC-T15
  source 1..59: mol_type = other DNA
ccggaaaccatgtgagtggtagccgtcaagaccggctaccactcacatggttttttttg

SEQ ID NO: 109 - DNA, 59 bp, synthetic construct
  misc_feature 1..59: note = LMNC-T16
  source 1..59: mol_type = other DNA
ccggaaccatgtgagtggtagccgctcaagacgcggctaccactcacatggtttttttg

SEQ ID NO: 110 - DNA, 59 bp, synthetic construct
  misc_feature 1..59: note = LMNC-T17
  source 1..59: mol_type = other DNA
ccggaacatgtgagtggtagccgcctcaagacggcggctaccactcacatgtttttttg

SEQ ID NO: 111 - DNA, 59 bp, synthetic construct
  misc_feature 1..59: note = LMNC-T18
  source 1..59: mol_type = other DNA
ccggaaatgtgagtggtagccgccgtcaagaccggcggctaccactcacattttttttg

SEQ ID NO: 112 - DNA, 59 bp, synthetic construct
  misc_feature 1..59: note = LMNC-T19
  source 1..59: mol_type = other DNA
ccggaatgtgagtggtagccgccgctcaagacgcggcggctaccactcacatttttttg

SEQ ID NO: 113 - DNA, 59 bp, synthetic construct; identical to SEQ ID NO: 75
  misc_feature 1..59: note = LMNA-C1
  source 1..59: mol_type = other DNA

SEQ ID NO: 114 - DNA, 59 bp, synthetic construct; identical to SEQ ID NO: 76
  misc_feature 1..59: note = LMNA-C2
  source 1..59: mol_type = other DNA

SEQ ID NO: 115 - DNA, 59 bp, synthetic construct
  misc_feature 1..59: note = LMNA-C3
  source 1..59: mol_type = other DNA
ccggaatgctccatcaccaccacggtcaagacccgtggtggtgatggagcatttttttg

SEQ ID NO: 116 - DNA, 59 bp, synthetic construct
  misc_feature 1..59: note = LMNA-C4
  source 1..59: mol_type = other DNA
ccggaagctccatcaccaccacggctcaagacgccgtggtggtgatggagctttttttg

SEQ ID NO: 117 - DNA, 59 bp, synthetic construct
  misc_feature 1..59: note = LMNA-C5
  source 1..59: mol_type = other DNA
ccggaactccatcaccaccacggcttcaagacagccgtggtggtgatggagtttttttg

SEQ ID NO: 118 - DNA, 59 bp, synthetic construct
  misc_feature 1..59: note = LMNA-C6
  source 1..59: mol_type = other DNA
ccggaatccatcaccaccacggctctcaagacgagccgtggtggtgatggatttttttg

SEQ ID NO: 119 - DNA, 59 bp, synthetic construct
  misc_feature 1..59: note = LMNA-C7
  source 1..59: mol_type = other DNA
ccggaaccatcaccaccacggctcctcaagacggagccgtggtggtgatggtttttttg

SEQ ID NO: 120 - DNA, 59 bp, synthetic construct
  misc_feature 1..59: note = LMNA-C8
  source 1..59: mol_type = other DNA
ccggaacatcaccaccacggctccctcaagacgggagccgtggtggtgatgtttttttg

SEQ ID NO: 121 - DNA, 59 bp, synthetic construct
  misc_feature 1..59: note = LMNA-C9
  source 1..59: mol_type = other DNA
ccggaaatcaccaccacggctcccatcaagactgggagccgtggtggtgattttttttg

SEQ ID NO: 122 - DNA, 59 bp, synthetic construct
  misc_feature 1..59: note = LMNA-C10
  source 1..59: mol_type = other DNA
ccggaatcaccaccacggctcccactcaagacgtgggagccgtggtggtgatttttttg

SEQ ID NO: 123 - DNA, 59 bp, synthetic construct
  misc_feature 1..59: note = LMNA-C11
  source 1..59: mol_type = other DNA
ccggaacaccaccacggctcccacttcaagacagtgggagccgtggtggtgtttttttg

SEQ ID NO: 124 - DNA, 59 bp, synthetic construct
  misc_feature 1..59: note = LMNA-C12
  source 1..59: mol_type = other DNA
ccggaaaccaccacggctcccactgtcaagaccagtgggagccgtggtggttttttttg

SEQ ID NO: 125 - DNA, 59 bp, synthetic construct
  misc_feature 1..59: note = LMNA-C13
  source 1..59: mol_type = other DNA
ccggaaccaccacggctcccactgctcaagacgcagtgggagccgtggtggtttttttg

SEQ ID NO: 126 - DNA, 59 bp, synthetic construct
  misc_feature 1..59: note = LMNA-C14
  source 1..59: mol_type = other DNA
ccggaacaccacggctcccactgcatcaagactgcagtgggagccgtggtgtttttttg

SEQ ID NO: 127 - DNA, 59 bp, synthetic construct
  misc_feature 1..59: note = LMNA-C15
  source 1..59: mol_type = other DNA
ccggaaaccacggctcccactgcagtcaagacctgcagtgggagccgtggttttttttg

SEQ ID NO: 128 - DNA, 59 bp, synthetic construct
  misc_feature 1..59: note = LMNA-C16
  source 1..59: mol_type = other DNA
ccggaaccacggctcccactgcagctcaagacgctgcagtgggagccgtggtttttttg

SEQ ID NO: 129 - DNA, 59 bp, synthetic construct
  misc_feature 1..59: note = LMNA-C17
  source 1..59: mol_type = other DNA
ccggaacacggctcccactgcagcatcaagactgctgcagtgggagccgtgtttttttg

SEQ ID NO: 130 - DNA, 59 bp, synthetic construct
  misc_feature 1..59: note = LMNA-C18
  source 1..59: mol_type = other DNA
ccggaaacggctcccactgcagcagtcaagacctgctgcagtgggagccgttttttttg

SEQ ID NO: 131 - DNA, 59 bp, synthetic construct
  misc_feature 1..59: note = LMNA-C19
  source 1..59: mol_type = other DNA
ccggaacggctcccactgcagcagctcaagacgctgctgcagtgggagccgtttttttg

SEQ ID NO: 132 - DNA, 59 bp, synthetic construct; identical to SEQ ID NO: 94
  misc_feature 1..59: note = LMNA-T1
  source 1..59: mol_type = other DNA

SEQ ID NO: 133 - DNA, 59 bp, synthetic construct; identical to SEQ ID NO: 95
  misc_feature 1..59: note = LMNA-T2
  source 1..59: mol_type = other DNA

SEQ ID NO: 134 - DNA, 59 bp, synthetic construct
  misc_feature 1..59: note = LMNA-T3
  source 1..59: mol_type = other DNA
ccggaatgctccatcaccaccatggtcaagacccatggtggtgatggagcatttttttg

SEQ ID NO: 135 - DNA, 59 bp, synthetic construct
  misc_feature 1..59: note = LMNA-T4
  source 1..59: mol_type = other DNA
ccggaagctccatcaccaccatggctcaagacgccatggtggtgatggagctttttttg

SEQ ID NO: 136 - DNA, 59 bp, synthetic construct
  misc_feature 1..59: note = LMNA-T5
  source 1..59: mol_type = other DNA
ccggaactccatcaccaccatggcttcaagacagccatggtggtgatggagtttttttg

SEQ ID NO: 137 - DNA, 59 bp, synthetic construct
  misc_feature 1..59: note = LMNA-T6
  source 1..59: mol_type = other DNA
ccggaatccatcaccaccatggctctcaagacgagccatggtggtgatggatttttttg

SEQ ID NO: 138 - DNA, 59 bp, synthetic construct
  misc_feature 1..59: note = LMNA-T7
  source 1..59: mol_type = other DNA
ccggaaccatcaccaccatggctcctcaagacggagccatggtggtgatggtttttttg

SEQ ID NO: 139 - DNA, 59 bp, synthetic construct
  misc_feature 1..59: note = LMNA-T8
  source 1..59: mol_type = other DNA
ccggaacatcaccaccatggctccctcaagacgggagccatggtggtgatgtttttttg

SEQ ID NO: 140 - DNA, 59 bp, synthetic construct
  misc_feature 1..59: note = LMNA-T9
  source 1..59: mol_type = other DNA
ccggaaatcaccaccatggctcccatcaagactgggagccatggtggtgattttttttg

SEQ ID NO: 141 - DNA, 59 bp, synthetic construct
  misc_feature 1..59: note = LMNA-T10
  source 1..59: mol_type = other DNA
ccggaatcaccaccatggctcccactcaagacgtgggagccatggtggtgatttttttg

SEQ ID NO: 142 - DNA, 59 bp, synthetic construct
  misc_feature 1..59: note = LMNA-T11
  source 1..59: mol_type = other DNA
ccggaacaccaccatggctcccacttcaagacagtgggagccatggtggtgtttttttg

SEQ ID NO: 143 - DNA, 59 bp, synthetic construct
  misc_feature 1..59: note = LMNA-T12
  source 1..59: mol_type = other DNA
ccggaaaccaccatggctcccactgtcaagaccagtgggagccatggtggttttttttg

SEQ ID NO: 144 - DNA, 59 bp, synthetic construct
  misc_feature 1..59: note = LMNA-T13
  source 1..59: mol_type = other DNA
ccggaaccaccatggctcccactgctcaagacgcagtgggagccatggtggtttttttg

SEQ ID NO: 145 - DNA, 59 bp, synthetic construct
  misc_feature 1..59: note = LMNA-T14
  source 1..59: mol_type = other DNA
ccggaacaccatggctcccactgcatcaagactgcagtgggagccatggtgtttttttg

SEQ ID NO: 146 - DNA, 59 bp, synthetic construct
  misc_feature 1..59: note = LMNA-T15
  source 1..59: mol_type = other DNA
ccggaaaccatggctcccactgcagtcaagacctgcagtgggagccatggttttttttg

SEQ ID NO: 147 - DNA, 59 bp, synthetic construct
  misc_feature 1..59: note = LMNA-T16
  source 1..59: mol_type = other DNA
ccggaaccatggctcccactgcagctcaagacgctgcagtgggagccatggtttttttg

SEQ ID NO: 148 - DNA, 59 bp, synthetic construct
  misc_feature 1..59: note = LMNA-T17
  source 1..59: mol_type = other DNA
ccggaacatggctcccactgcagcatcaagactgctgcagtgggagccatgtttttttg

SEQ ID NO: 149 - DNA, 59 bp, synthetic construct
  misc_feature 1..59: note = LMNA-T18
  source 1..59: mol_type = other DNA
ccggaaatggctcccactgcagcagtcaagacctgctgcagtgggagccattttttttg

SEQ ID NO: 150 - DNA, 59 bp, synthetic construct
  misc_feature 1..59: note = LMNA-T19
  source 1..59: mol_type = other DNA
ccggaatggctcccactgcagcagctcaagacgctgctgcagtgggagccatttttttg

SEQ ID NO: 151 - RNA, 37 nt, synthetic construct
  misc_feature 1..37: note = antisense of C variant of LMNC
  source 1..37: mol_type = other RNA
gcggcggctaccactcacgtggtggtgatggagcagg

SEQ ID NO: 152 - RNA, 37 nt, synthetic construct
  misc_feature 1..37: note = antisense of U variant of LMNC
  source 1..37: mol_type = other RNA
gcggcggctaccactcacatggtggtgatggagcagg

SEQ ID NO: 153 - RNA, 37 nt, synthetic construct
  misc_feature 1..37: note = antisense LMNA C variant
  source 1..37: mol_type = other RNA
gctgctgcagtgggagccgtggtggtgatggagcagg

SEQ ID NO: 154 - RNA, 37 nt, synthetic construct
  misc_feature 1..37: note = antisense of U variant of LMNA
  source 1..37: mol_type = other RNA
gctgctgcagtgggagccatggtggtgatggagcagg

SEQ ID NO: 155 - DNA, 12 bp, synthetic construct
  misc_feature 1..12: note = shRNA p. 3, 26 and 29
  source 1..12: mol_type = other DNA
aattcaaaaaaa

SEQ ID NO: 156 - RNA, 19 nt, synthetic construct
  misc_feature 1..19: note = siRNA-C10
  source 1..19: mol_type = other RNA
agtggtggtgcactcacca

SEQ ID NO: 157 - RNA, 19 nt, synthetic construct
  misc_feature 1..19: note = siRNA-C11
  source 1..19: mol_type = other RNA
gtggtggtgcactcaccat

SEQ ID NO: 158 - RNA, 19 nt, synthetic construct
  misc_feature 1..19: note = siRNA-C12
  source 1..19: mol_type = other RNA
tggtggtgcactcaccatc

SEQ ID NO: 159 - RNA, 19 nt, synthetic construct
  misc_feature 1..19: note = siRNA-C13
  source 1..19: mol_type = other RNA
ggtggtgcactcaccatcg

SEQ ID NO: 160 - RNA, 19 nt, synthetic construct
  misc_feature 1..19: note = siRNA-T9
  source 1..19: mol_type = other RNA
tagtggtggtacactcacc

SEQ ID NO: 161 - RNA, 19 nt, synthetic construct
  misc_feature 1..19: note = siRNA-T10
  source 1..19: mol_type = other RNA
agtggtggtacactcacca

SEQ ID NO: 162 - RNA, 19 nt, synthetic construct
  misc_feature 1..19: note = siRNA-T11
  source 1..19: mol_type = other RNA
gtggtggtacactcaccat

SEQ ID NO: 163 - RNA, 19 nt, synthetic construct
  misc_feature 1..19: note = siRNA-T12
  source 1..19: mol_type = other RNA
tggtggtacactcaccatc

SEQ ID NO: 164 - RNA, 19 nt, synthetic construct
  misc_feature 1..19: note = siRNA-T13
  source 1..19: mol_type = other RNA
ggtggtacactcaccatcg

SEQ ID NO: 165 - RNA, 19 nt, synthetic construct
  misc_feature 1..19: note = siRNA-T14
  source 1..19: mol_type = other RNA
gtggtacactcaccatcgg

SEQ ID NO: 166 - RNA, 45 nt, synthetic construct
  misc_feature 1..44: note = Lamin A Fig. 1
  source 1..45: mol_type = other RNA
gatgacctgctccatcaccaccacggctcccactgcagcagctcg

SEQ ID NO: 167 - RNA, 19 nt, synthetic construct
  misc_feature 1..19: note = siRNA-C10
  source 1..19: mol_type = other RNA
agtggtggtgccgagggtg

SEQ ID NO: 168 - RNA, 19 nt, synthetic construct
  misc_feature 1..19: note = siRNA-C11
  source 1..19: mol_type = other RNA
gtggtggtgccgagggtga

SEQ ID NO: 169 - RNA, 19 nt, synthetic construct
  misc_feature 1..19: note = siRNA-C12
  source 1..19: mol_type = other RNA
tggtggtgccgagggtgac

SEQ ID NO: 170 - RNA, 19 nt, synthetic construct
  misc_feature 1..19: note = siRNA-C13
  source 1..19: mol_type = other RNA
ggtggtgccgagggtgacg

SEQ ID NO: 171 - RNA, 19 nt, synthetic construct
  misc_feature 1..19: note = siRNA-C14
  source 1..19: mol_type = other RNA
gtggtgccgagggtgacgt

SEQ ID NO: 172 - RNA, 19 nt, synthetic construct
  misc_feature 1..19: note = siRNA-T9
  source 1..19: mol_type = other RNA
tagtggtggtaccgagggt

SEQ ID NO: 173 - RNA, 19 nt, synthetic construct
  misc_feature 1..19: note = siRNA-T10
  source 1..19: mol_type = other RNA
agtggtggtaccgagggtg

SEQ ID NO: 174 - RNA, 19 nt, synthetic construct
  misc_feature 1..19: note = siRNA-T11
  source 1..19: mol_type = other RNA
gtggtggtaccgagggtga

SEQ ID NO: 175 - RNA, 19 nt, synthetic construct
  misc_feature 1..19: note = siRNA-T12
  source 1..19: mol_type = other RNA
tggtggtaccgagggtgac

SEQ ID NO: 176 - RNA, 19 nt, synthetic construct
  misc_feature 1..19: note = siRNA-T14
  source 1..19: mol_type = other RNA
gtggtaccgagggtgacgt

SEQ ID NO: 177 - RNA, 19 nt, synthetic construct
  misc_feature 1..19: note = siRNA-T17
  source 1..19: mol_type = other RNA
gtaccgagggtgacgtcgt

SEQ ID NO: 178 - RNA, 19 nt, synthetic construct
  misc_feature 1..19: note = siRNA-C17
  source 1..19: mol_type = other RNA
gtgccgagggtgacgtcgt

SEQ ID NO: 179 - DNA, 19 bp, synthetic construct
  source 1..19: mol_type = other DNA
cacgtgagtggtagccgcc

SEQ ID NO: 180 - DNA, 19 bp, synthetic construct
  source 1..19: mol_type = other DNA
ggcggctaccactcacgtg

SEQ ID NO: 181 - DNA, 19 bp, synthetic construct
  source 1..19: mol_type = other DNA
caccaccacgtgagaggta

SEQ ID NO: 182 - DNA, 19 bp, synthetic construct
  source 1..19: mol_type = other DNA
tacctctcacgtggtggtg

SEQ ID NO: 183 - DNA, 19 bp, synthetic construct
  source 1..19: mol_type = other DNA
caccaccacgtgactggta

SEQ ID NO: 184 - DNA, 19 bp, synthetic construct
  source 1..19: mol_type = other DNA
taccagtcacgtggtggtg

SEQ ID NO: 185 - DNA, 19 bp, synthetic construct
  source 1..19: mol_type = other DNA
caccaccacgtgtgtggta

SEQ ID NO: 186 - DNA, 19 bp, synthetic construct
  source 1..19: mol_type = other DNA
taccacacacgtggtggtg

SEQ ID NO: 187 - DNA, 19 bp, synthetic construct
  source 1..19: mol_type = other DNA
caccacgacgtgagtggta

SEQ ID NO: 188 - DNA, 19 bp, synthetic construct
  source 1..19: mol_type = other DNA
taccactcacgtcgtggtg

SEQ ID NO: 189 - DNA, 19 bp, synthetic construct
  source 1..19: mol_type = other DNA
cagcaccacgtgagtggta

SEQ ID NO: 190 - DNA, 19 bp, synthetic construct
  source 1..19: mol_type = other DNA
taccactcacgtggtgctg

SEQ ID NO: 191 - DNA, 19 bp, synthetic construct
  source 1..19: mol_type = other DNA
ccaccacgtgagtgctagc

SEQ ID NO: 192 - DNA, 19 bp, synthetic construct
  source 1..19: mol_type = other DNA
gctagcactcacgtggtgg

SEQ ID NO: 193 - DNA, 19 bp, synthetic construct
  source 1..19: mol_type = other DNA
ccaccacgtgagtcgtagc

SEQ ID NO: 194 - DNA, 19 bp, synthetic construct
  source 1..19: mol_type = other DNA
gctacgactcacgtggtgg

SEQ ID NO: 195 - DNA, 19 bp, synthetic construct
  source 1..19: mol_type = other DNA
ccaccacgtgagaggtagc

SEQ ID NO: 196 - DNA, 19 bp, synthetic construct
  source 1..19: mol_type = other DNA
gctacctctcacgtggtgg

SEQ ID NO: 197 - DNA, 19 bp, synthetic construct
  source 1..19: mol_type = other DNA
ccatgtgagtggtagccgc

SEQ ID NO: 198 - DNA, 19 bp, synthetic construct
  source 1..19: mol_type = other DNA
gcggctaccactcacatgg

SEQ ID NO: 199 - DNA, 19 bp, synthetic construct
  source 1..19: mol_type = other DNA
catgtgagtggtagccgcc

SEQ ID NO: 200 - DNA, 19 bp, synthetic construct
  source 1..19: mol_type = other DNA
ggcggctaccactcacatg

SEQ ID NO: 201 - DNA, 19 bp, synthetic construct
  source 1..19: mol_type = other DNA
caccatgtgagtggaagcc

SEQ ID NO: 202 - DNA, 19 bp, synthetic construct
  source 1..19: mol_type = other DNA
ggcttccactcacatggtg

SEQ ID NO: 203 - DNA, 19 bp, synthetic construct
  source 1..19: mol_type = other DNA
caccatgtgagtgctagcc

SEQ ID NO: 204 - DNA, 19 bp, synthetic construct
  source 1..19: mol_type = other DNA
ggctagcactcacatggtg

SEQ ID NO: 205 - DNA, 19 bp, synthetic construct
  source 1..19: mol_type = other DNA
caccatgtgagtcgtagcc

SEQ ID NO: 206 - DNA, 19 bp, synthetic construct
  source 1..19: mol_type = other DNA
ggctacgactcacatggtg

SEQ ID NO: 207 - DNA, 19 bp, synthetic construct
  source 1..19: mol_type = other DNA
caccatgtgtgtggtagcc

SEQ ID NO: 208 - DNA, 19 bp, synthetic construct
  source 1..19: mol_type = other DNA
ggctaccacacacatggtg

SEQ ID NO: 209 - DNA, 19 bp, synthetic construct
  source 1..19: mol_type = other DNA
caccatgtcagtggtagcc

SEQ ID NO: 210 - DNA, 19 bp, synthetic construct
  source 1..19: mol_type = other DNA
ggctaccactgacatggtg

SEQ ID NO: 211 - DNA, 19 bp, synthetic construct
  source 1..19: mol_type = other DNA
cagcatgtgagtggtagcc

SEQ ID NO: 212 - DNA, 19 bp, synthetic construct
  source 1..19: mol_type = other DNA
ggctaccactcacatgctg

SEQ ID NO: 213 - DNA, 19 bp, synthetic construct
  source 1..19: mol_type = other DNA
ccatgtgagtggtagccgc

SEQ ID NO: 214 - DNA, 19 bp, synthetic construct
  source 1..19: mol_type = other DNA
gcggctaccactcacatgg

SEQ ID NO: 215 - DNA, 19 bp, synthetic construct
  source 1..19: mol_type = other DNA
cacggctcccactggagca

SEQ ID NO: 216 - DNA, 19 bp, synthetic construct
  source 1..19: mol_type = other DNA
tgctccagtgggagccgtg

SEQ ID NO: 217 - DNA, 19 bp, synthetic construct
  source 1..19: mol_type = other DNA
cacggctcccactccagca

SEQ ID NO: 218 - DNA, 19 bp, synthetic construct
  source 1..19: mol_type = other DNA
tgctggagtgggagccgtg

SEQ ID NO: 219 - DNA, 19 bp, synthetic construct
  source 1..19: mol_type = other DNA
cacggctcccacagcagca

SEQ ID NO: 220 - DNA, 19 bp, synthetic construct
  source 1..19: mol_type = other DNA
tgctgctgtgggagccgtg

SEQ ID NO: 221 - DNA, 19 bp, synthetic construct
  source 1..19: mol_type = other DNA
cacggctccgactgcagca

SEQ ID NO: 222 - DNA, 19 bp, synthetic construct
  source 1..19: mol_type = other DNA
tgctgcagtcggagccgtg

SEQ ID NO: 223 - DNA, 19 bp, synthetic construct
  source 1..19: mol_type = other DNA
cacggctcgcactgcagca

SEQ ID NO: 224 - DNA, 19 bp, synthetic construct
  source 1..19: mol_type = other DNA
tgctgcagtgcgagccgtg

SEQ ID NO: 225 - DNA, 19 bp, synthetic construct
  source 1..19: mol_type = other DNA
catggctcccactggagca

SEQ ID NO: 226 - DNA, 19 bp, synthetic construct
  source 1..19: mol_type = other DNA
tgctccagtgggagccatg

SEQ ID NO: 227 - DNA, 19 bp, synthetic construct
  source 1..19: mol_type = other DNA
catggctcccactccagca

SEQ ID NO: 228 - DNA, 19 bp, synthetic construct
  source 1..19: mol_type = other DNA
tgctggagtgggagccatg

SEQ ID NO: 229 - DNA, 19 bp, synthetic construct
  source 1..19: mol_type = other DNA
catggctcccacagcagca

SEQ ID NO: 230 - DNA, 19 bp, synthetic construct
  source 1..19: mol_type = other DNA
tgctgctgtgggagccatg

SEQ ID NO: 231 - DNA, 19 bp, synthetic construct
  source 1..19: mol_type = other DNA
catggctccgactgcagca

SEQ ID NO: 232 - DNA, 19 bp, synthetic construct
  source 1..19: mol_type = other DNA
tgctgcagtcggagccatg

SEQ ID NO: 233 - DNA, 19 bp, synthetic construct
  source 1..19: mol_type = other DNA
catggctcgcactgcagca

SEQ ID NO: 234 - DNA, 19 bp, synthetic construct
  source 1..19: mol_type = other DNA
tgctgcagtgcgagccatg

SEQ ID NO: 235 - DNA, 59 bp, synthetic construct
  source 1..59: mol_type = other DNA
ccggaacacggctcccactggagcatcaagactgctccagtgggagccgtgtttttttg

SEQ ID NO: 236 - DNA, 59 bp, synthetic construct
  source 1..59: mol_type = other DNA
aattcaaaaaaacacggctcccactggagcagtcttgatgctccagtgggagccgtgtt

SEQ ID NO: 237 - DNA, 59 bp, synthetic construct
  source 1..59: mol_type = other DNA
ccggaacacggctcccactccagcatcaagactgctggagtgggagccgtgtttttttg

SEQ ID NO: 238 - DNA, 59 bp, synthetic construct
  source 1..59: mol_type = other DNA
aattcaaaaaaacacggctcccactccagcagtcttgatgctggagtgggagccgtgtt

SEQ ID NO: 239 - DNA, 59 bp, synthetic construct
  source 1..59: mol_type = other DNA
ccggaacacggctcccacagcagcatcaagactgctgctgtgggagccgtgtttttttg

SEQ ID NO: 240 - DNA, 59 bp, synthetic construct
  source 1..59: mol_type = other DNA
aattcaaaaaaacacggctcccacagcagcagtcttgatgctgctgtgggagccgtgtt

SEQ ID NO: 241 - DNA, 59 bp, synthetic construct
  source 1..59: mol_type = other DNA
ccggaacacggctccgactgcagcatcaagactgctgcagtcggagccgtgtttttttg

SEQ ID NO: 242 - DNA, 59 bp, synthetic construct
  source 1..59: mol_type = other DNA
aattcaaaaaaacacggctccgactgcagcagtcttgatgctgcagtcggagccgtgtt

SEQ ID NO: 243 - DNA, 59 bp, synthetic construct
  source 1..59: mol_type = other DNA
ccggaacacggctcgcactgcagcatcaagactgctgcagtgcgagccgtgtttttttg

SEQ ID NO: 244 - DNA, 59 bp, synthetic construct
  source 1..59: mol_type = other DNA
aattcaaaaaaacacggctcgcactgcagcagtcttgatgctgcagtgcgagccgtgtt

SEQ ID NO: 245 - DNA, 59 bp, synthetic construct
  source 1..59: mol_type = other DNA
ccggaacatggctcccactggagcatcaagactgctccagtgggagccatgtttttttg

SEQ ID NO: 246 - DNA, 59 bp, synthetic construct
  source 1..59: mol_type = other DNA
aattcaaaaaaacatggctcccactggagcagtcttgatgctccagtgggagccatgtt

SEQ ID NO: 247 - DNA, 59 bp, synthetic construct
  source 1..59: mol_type = other DNA
ccggaacatggctcccactccagcatcaagactgctggagtgggagccatgtttttttg

SEQ ID NO: 248 - DNA, 59 bp, synthetic construct
  source 1..59: mol_type = other DNA
aattcaaaaaaacatggctcccactccagcagtcttgatgctggagtgggagccatgtt

SEQ ID NO: 249 - DNA, 59 bp, synthetic construct
  source 1..59: mol_type = other DNA
ccggaacatggctcccacagcagcatcaagactgctgctgtgggagccatgtttttttg

SEQ ID NO: 250 - DNA, 59 bp, synthetic construct
  source 1..59: mol_type = other DNA
aattcaaaaaaacatggctcccacagcagcagtcttgatgctgctgtgggagccatgtt

SEQ ID NO: 251 - DNA, 59 bp, synthetic construct
  source 1..59: mol_type = other DNA
ccggaacatggctccgactgcagcatcaagactgctgcagtcggagccatgtttttttg

SEQ ID NO: 252 - DNA, 59 bp, synthetic construct
  source 1..59: mol_type = other DNA
aattcaaaaaaacatggctccgactgcagcagtcttgatgctgcagtcggagccatgtt

SEQ ID NO: 253 - DNA, 59 bp, synthetic construct
  source 1..59: mol_type = other DNA
ccggaacatggctcgcactgcagcatcaagactgctgcagtgcgagccatgtttttttg

SEQ ID NO: 254 - DNA, 59 bp, synthetic construct
  source 1..59: mol_type = other DNA
aattcaaaaaaacatggctcgcactgcagcagtcttgatgctgcagtgcgagccatgtt

SEQ ID NO: 255 - DNA, 59 bp, synthetic construct; identical to SEQ ID NO: 91
  source 1..59: mol_type = other DNA

SEQ ID NO: 256 - DNA, 59 bp, synthetic construct
  source 1..59: mol_type = other DNA
aattcaaaaaaacacgtgagtggtagccgccgtcttgaggcggctaccactcacgtgtt

SEQ ID NO: 257 - DNA, 59 bp, synthetic construct; identical to SEQ ID NO: 109
  source 1..59: mol_type = other DNA

SEQ ID NO: 258 - DNA, 59 bp, synthetic construct
  source 1..59: mol_type = other DNA
aattcaaaaaaaccatgtgagtggtagccgcgtcttgagcggctaccactcacatggtt

SEQ ID NO: 259 - DNA, 59 bp, synthetic construct; identical to SEQ ID NO: 110
  source 1..59: mol_type = other DNA

SEQ ID NO: 260 - DNA, 59 bp, synthetic construct
  source 1..59: mol_type = other DNA
aattcaaaaaaacatgtgagtggtagccgccgtcttgaggcggctaccactcacatgtt

SEQ ID NO: 261 - DNA, 59 bp, synthetic construct
  source 1..59: mol_type = other DNA
ccggaacaccaccacgtgagaggtatcaagactacctctcacgtggtggtgtttttttg

SEQ ID NO: 262 - DNA, 59 bp, synthetic construct
  source 1..59: mol_type = other DNA
aattcaaaaaaacaccaccacgtgagaggtagtcttgatacctctcacgtggtggtgtt

SEQ ID NO: 263 - DNA, 59 bp, synthetic construct
  source 1..59: mol_type = other DNA
ccggaacaccaccacgtgactggtatcaagactaccagtcacgtggtggtgtttttttg

SEQ ID NO: 264 - DNA, 59 bp, synthetic construct
  source 1..59: mol_type = other DNA
aattcaaaaaaacaccaccacgtgactggtagtcttgataccagtcacgtggtggtgtt

SEQ ID NO: 265 - DNA, 59 bp, synthetic construct
  source 1..59: mol_type = other DNA
ccggaacaccaccacgtgtgtggtatcaagactaccacacacgtggtggtgtttttttg

SEQ ID NO: 266 - DNA, 59 bp, synthetic construct
  source 1..59: mol_type = other DNA
aattcaaaaaaacaccaccacgtgtgtggtagtcttgataccacacacgtggtggtgtt

SEQ ID NO: 267 - DNA, 59 bp, synthetic construct
  source 1..59: mol_type = other DNA
ccggaacaccacgacgtgagtggtatcaagactaccactcacgtcgtggtgtttttttg

SEQ ID NO: 268 - DNA, 59 bp, synthetic construct
  source 1..59: mol_type = other DNA
aattcaaaaaaacaccacgacgtgagtggtagtcttgataccactcacgtcgtggtgtt

SEQ ID NO: 269 - DNA, 59 bp, synthetic construct
  source 1..59: mol_type = other DNA
ccggaacagcaccacgtgagtggtatcaagactaccactcacgtggtgctgtttttttg

SEQ ID NO: 270 - DNA, 59 bp, synthetic construct
  source 1..59: mol_type = other DNA
aattcaaaaaaacagcaccacgtgagtggtagtcttgataccactcacgtggtgctgtt

SEQ ID NO: 271 - DNA, 59 bp, synthetic construct
  source 1..59: mol_type = other DNA
ccggaaccaccacgtgagtgctagctcaagacgctagcactcacgtggtggtttttttg

SEQ ID NO: 272 - DNA, 59 bp, synthetic construct
  source 1..59: mol_type = other DNA
aattcaaaaaaaccaccacgtgagtgctagcgtcttgagctagcactcacgtggtggtt

SEQ ID NO: 273 - DNA, 59 bp, synthetic construct
  source 1..59: mol_type = other DNA
ccggaaccaccacgtgagtcgtagctcaagacgctacgactcacgtggtggtttttttg

SEQ ID NO: 274 - DNA, 59 bp, synthetic construct
  source 1..59: mol_type = other DNA
aattcaaaaaaaccaccacgtgagtcgtagcgtcttgagctacgactcacgtggtggtt

SEQ ID NO: 275 - DNA, 59 bp, synthetic construct
  source 1..59: mol_type = other DNA
ccggaaccaccacgtgagaggtagctcaagacgctacctctcacgtggtggtttttttg

SEQ ID NO: 276 - DNA, 59 bp, synthetic construct
  source 1..59: mol_type = other DNA
aattcaaaaaaaccaccacgtgagaggtagcgtcttgagctacctctcacgtggtggtt

SEQ ID NO: 277 - DNA, 59 bp, synthetic construct
  source 1..59: mol_type = other DNA
ccggaaccaccacgagagtggtagctcaagacgctaccactctcgtggtggtttttttg

SEQ ID NO: 278 - DNA, 59 bp, synthetic construct
  source 1..59: mol_type = other DNA
aattcaaaaaaaccaccacgagagtggtagcgtcttgagctaccactctcgtggtggtt

SEQ ID NO: 279 - DNA, 59 bp, synthetic construct
  source 1..59: mol_type = other DNA
ccggaacctccacgtgagtggtagctcaagacgctaccactcacgtggaggtttttttg

SEQ ID NO: 280 - DNA, 59 bp, synthetic construct
  source 1..59: mol_type = other DNA
aattcaaaaaaacctccacgtgagtggtagcgtcttgagctaccactcacgtggaggtt

SEQ ID NO: 281 - DNA, 59 bp, synthetic construct
  source 1..59: mol_type = other DNA
ccggaacaccatgtgagtggaagcctcaagacggcttccactcacatggtgtttttttg

SEQ ID NO: 282 - DNA, 59 bp, synthetic construct
  source 1..59: mol_type = other DNA
aattcaaaaaaacaccatgtgagtggaagccgtcttgaggcttccactcacatggtgtt

SEQ ID NO: 283 - DNA, 59 bp, synthetic construct
  source 1..59: mol_type = other DNA
ccggaacaccatgtgagtgctagcctcaagacggctagcactcacatggtgtttttttg

SEQ ID NO: 284 - DNA, 59 bp, synthetic construct
  source 1..59: mol_type = other DNA
aattcaaaaaaacaccatgtgagtgctagccgtcttgaggctagcactcacatggtgtt

SEQ ID NO: 285 - DNA, 59 bp, synthetic construct
  source 1..59: mol_type = other DNA
ccggaacaccatgtgagtcgtagcctcaagacggctacgactcacatggtgtttttttg

SEQ ID NO: 286 - DNA, 59 bp, synthetic construct
  source 1..59: mol_type = other DNA
aattcaaaaaaacaccatgtgagtcgtagccgtcttgaggctacgactcacatggtgtt

SEQ ID NO: 287 - DNA, 59 bp, synthetic construct
  source 1..59: mol_type = other DNA
ccggaacaccatgtgtgtggtagcctcaagacggctaccacacacatggtgtttttttg

SEQ ID NO: 288 - DNA, 59 bp, synthetic construct
  source 1..59: mol_type = other DNA
aattcaaaaaaacaccatgtgtgtggtagccgtcttgaggctaccacacacatggtgtt

SEQ ID NO: 289 - DNA, 59 bp, synthetic construct
  source 1..59: mol_type = other DNA
ccggaacaccatgtcagtggtagcctcaagacggctaccactgacatggtgtttttttg

SEQ ID NO: 290 - DNA, 59 bp, synthetic construct
  source 1..59: mol_type = other DNA
aattcaaaaaaacaccatgtcagtggtagccgtcttgaggctaccactcagatggtgtt

SEQ ID NO: 291 - DNA, 59 bp, synthetic construct
  source 1..59: mol_type = other DNA
ccggaacagcatgtgagtggtagcctcaagacggctaccactcacatgctgtttttttg

SEQ ID NO: 292 - DNA, 59 bp, synthetic construct
  source 1..59: mol_type = other DNA
aattcaaaaaaacagcatgtgagtggtagccgtcttgaggctaccactcacatgctgtt